(19) United States
(12) Patent Application Publication
(10) Pub. No.: US 2007/0219370 A1
(43) Pub. Date: Sep. 20, 2007

(54) PROCESS FOR PREPARING N-(2-CHLORO-6-METHYLPHENYL)-2-[[6-[4-(2-HYDROXYETHYL)-1-PIPERAZINYL]-2-METHYL-4-PYRIMIDINYL]AMINO] -5-THIAZOLECARBOXAMIDE AND RELATED METABOLITES THEREOF

(75) Inventors: Jung-Hui Sun, Hockessin, DE (US); Jianqing Li, Guilford, CT (US); Bang-Chi Chen, Plainsboro, NJ (US); Daniel Smith, Hamden, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/685,897

(22) Filed: Mar. 14, 2007

Related U.S. Application Data
(60) Provisional application No. 60/782,622, filed on Mar. 15, 2006.

(51) Int. Cl. C07D 417/14

(52) U.S. Cl. 544/295

(57) ABSTRACT

The present invention is directed to process for the preparation of metabolites as well as the parent compound of N-(2-chloro-6-methylphenyl)-2-[[6-[4-(2-hydroxyethyl)-1-piperazinyl]-2-methyl-4-pyrimidinyl] amino]-5-thiazolecarboxamide, the compound of formula (I).

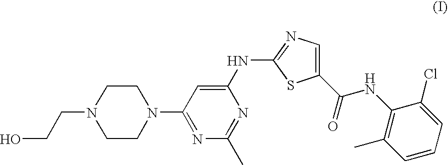

### CROSS-REFERENCE TO RELATED APPLICATION

This application claims the benefit of U.S. Provisional Application No. 60/782,622, filed on Mar. 15, 2006, which is herein incorporated by reference in its entirety.

### FIELD OF THE INVENTION

The invention relates to new processes for the preparation of compound of formula (II), N-(2-chloro-6-(hydroxymethyl)phenyl)-2-(6-(4-(2-hydroxyethyl)piperazin-1-yl)-2-methylpyrimidin-4-ylamino)thiazole-5-carboxamide, a hydroxyl metabolite of compound of formula (I). The invention also relates to new processes for the preparation of compound of formula (I) itself, as well as compound of formula (IV), the carboxyl metabolite of (I), N-(2-chloro-6-methylphenyl)-2-[[6-[4-(2-hydroxyethyl)-1-piperazinyl]-2-methyl-4-pyrimidinyl]amino]-5-thiazolecarboxamide useful in the treatment of oncological and immunological disorders.

### BACKGROUND OF THE INVENTION

The compound of formula (I), N-(2-chloro-6-methylphenyl)-2-[[6-[4-(2-hydroxyethyl)-1-piperazinyl]-2-methyl-4-pyrimidinyl]amino]-5-thiazolecarboxamide, is a protein tyrosine kinase inhibitor, a Src Kinase inhibitor, and is useful in the treatment of immunologic and oncological diseases. The compound of formula (II), N-(2-chloro-6-(hydroxymethyl)phenyl)-2-(6-(4-(2-hydroxyethyl)piperazin-1-yl)-2-methylpyrimidin-4-ylamino)thiazole-5-carboxamide, is a hydroxy metabolite of compound of formula (I), N-(2-chloro-6-methylphenyl)-2-[[6-[4-(2-hydroxyethyl)-1-piperazinyl]-2-methyl-4-pyrimidinyl]amino]-5-thiazolecarboxamide. The compound of formula (IV), is a carboxyl metabolite of compound of formula (I).

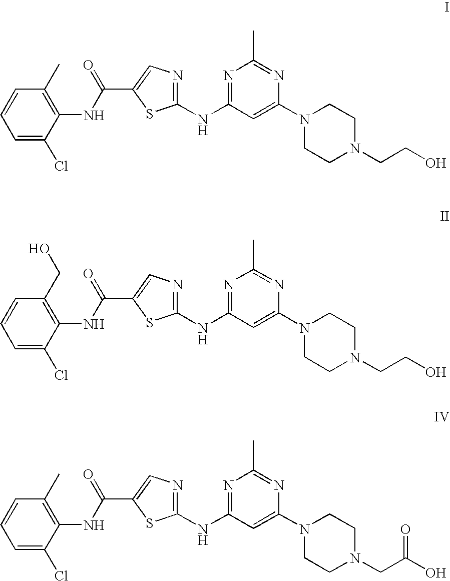

The compound of formula (I) and its preparation have been previously described in U.S. Pat. No. 6,596,746, issued Jul. 22, 2003, and US Patent applications US2005/0176965 A1, published Aug. 11, 2005, and US2006/0004067 A1, published Jan. 5, 2006.

The compounds of formula (II), and (IV) are metabolites of the compound of formula (I). The compounds and their utility are described in copending patent application, (U.S. Ser. No. 11/376,665, filed Mar. 15, 2006, which claims priority to U.S. Provisional application, 60/661,777, filed Mar. 15, 2005 and to U.S. Provisional application No. 60/728,732, filed Oct. 20, 2005.)

### DETAILED DESCRIPTION OF EMBODIMENTS OF THE INVENTION

The disclosure is directed to new processes for the preparation of compound of formula (II), N-(2-chloro-4-hydroxy-6-methylphenyl)-2-(6-(4-(2-hydroxyethyl)piperazin-1-yl)-2-methylpyrimidin-4-ylamino)thiazole-5-carboxamide, the hydroxyl metabolites of compound of formula (I), N-(2-chloro-6-methylphenyl)-2-[[6-[4-(2-hydroxyethyl)-1-piperazinyl]-2-methyl-4-pyrimidinyl]amino]-5-thiazolecarboxamide. The disclosure is also directed to new processes for the preparation of compound of formula (I) itself, as well as compound of formula (IV), the carboxyl metabolite of (I), N-(2-chloro-6-methylphenyl)-2-[[6-[4-(2-hydroxyethyl)-1-piperazinyl]-2-methyl-4-pyrimidinyl]amino]-5-thiazolecarboxamide,

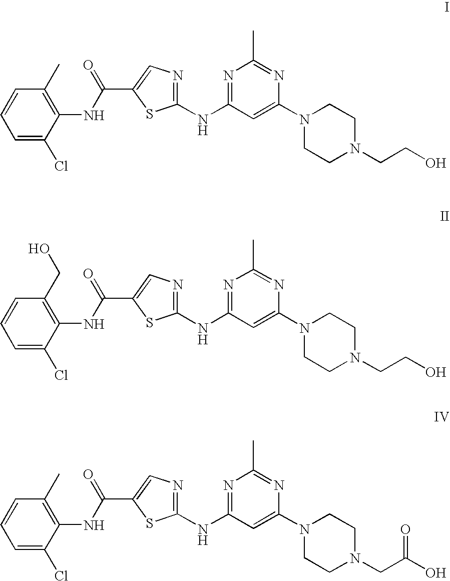

or pharmaceutically acceptable salt forms thereof

In one embodiment, a process is provided for preparing a compound of formula (Ia)

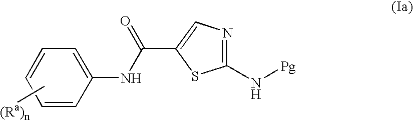

wherein R^{a} is selected from H, alkyl, halo, alkoxyl, C(O)OH, C(O)O-alkyl, alkyl-OC(O)—R¹, hydroxyalkyl and/or suitable protecting groups thereof;
n is selected from 0, 1, 2 and 3;

### R¹ is selected from hydrogen, alkyl and aryl;

including the steps of reacting a compound of formula 6

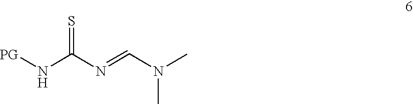

where PG is a nitrogen protecting group, with a compound of formula (A)

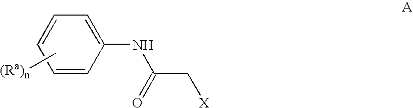

wherein X is selected from Cl, Br, I, mesylate, and tosylate; to provide the compound of formula (Ia).

In one embodiment, a process is provided for preparing a compound of formula (Ia)

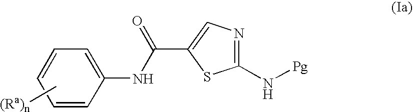

wherein R^{a} is selected from H, alkyl, halo, alkoxyl, C(O)OH, C(O)O-alkyl, alkyl-OC(O)—R¹, and/or suitable protecting groups thereof,
n is selected from 0, 1, 2 and 3;

### R¹ is selected from hydrogen, alkyl and aryl;

including the steps of reacting a compound of formula 6

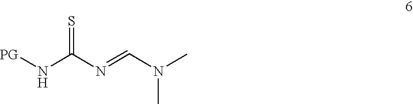

where PG is a nitrogen protecting group, with a compound of formula

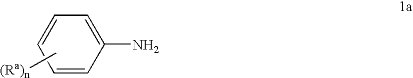

to provide the compound of formula (Ia).

In another embodiment, a process is provided for preparing a compound of formula (IIa)

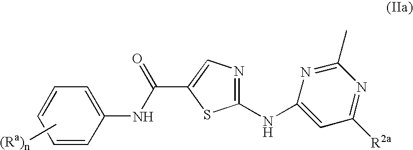

wherein R^{a} and n are as described above and R^{2a} is selected from halo,

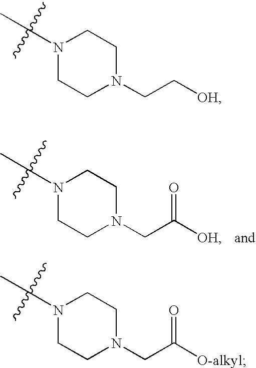

including the steps of reacting the compound of formula 1a

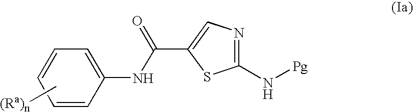

wherein R^{a}, n and Pg are as defined, with a deprotecting group to remove the Pg, followed by reaction with a compound of formula 2a

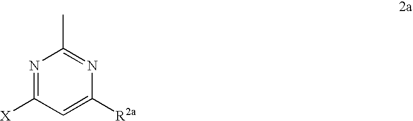

wherein X is selected from Cl, Br, and I,
to give the compound of formula IIa.

In one aspect, a process is provided for preparing a compound of formula II

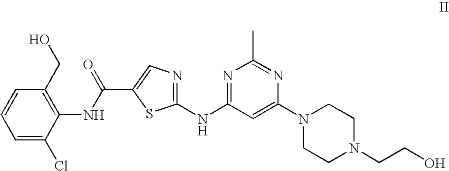

which includes the steps of
a) reacting a compound with a formula 1

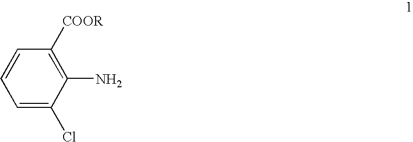

where R is selected from hydrogen, alkyl and aryl, with a reducing reagent to give a compound of formula 2

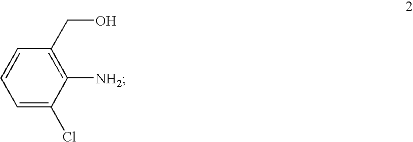

b) reacting the compound of formula 2 with a compound of formula 3

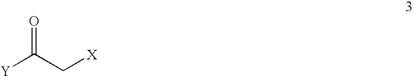

where X and Y is independently selected from Cl, Br, and I, in the presence of a base to give a compound of formula 4

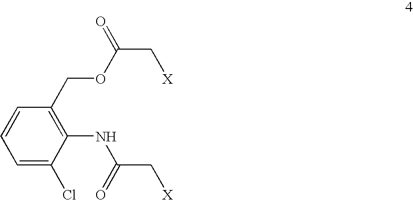

where X is as defined above;
c) reacting the compound of formula 4 with a base to give a compound of formula 5

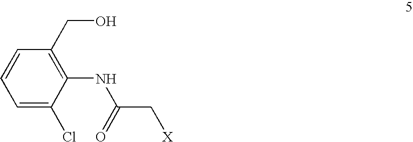

where X is as defined above;
d) reacting the compound of formula 5 with a compound of formula 6

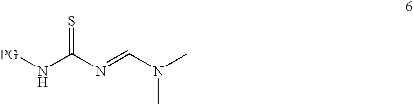

where PG is a nitrogen protecting group, to give a compound of formula 7

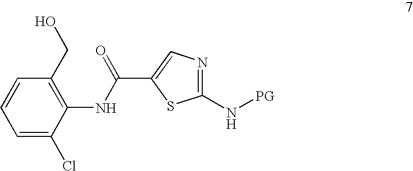

where PG is as defined above;
e) reacting the compound of formula 7 with a compound of formula 8

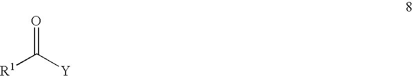

where Y is as defined above and R¹ is selected from hydrogen, alkyl and aryl, to give a compound of formula 9

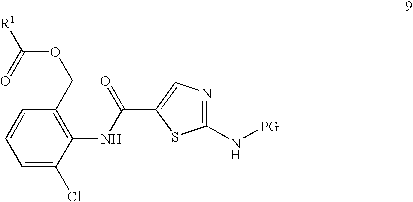

where R¹ and PG are as defined above;
g) treating the compound of formula 9 with a deprotecting reagent to give a compound of formula 10

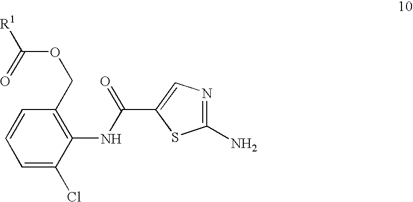

where R¹ is as defined above;
h) reacting the compound of formula 10 with a compound of formula 11

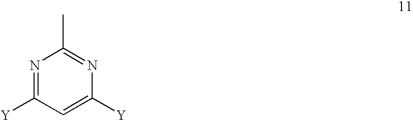

where X is as defined above, in the presence of a base to give compound of formula 12

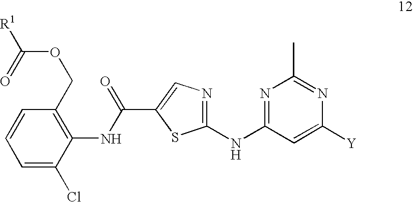

where X is as defined above;
i) reacting the compound of formula 12 with a base to give compound of formula 13

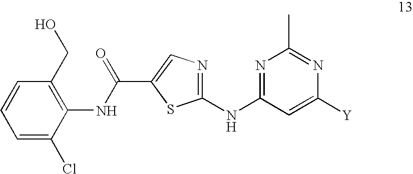

where X is as defined above;
j) and reacting the compound of formula 13 with a compound of formula 14

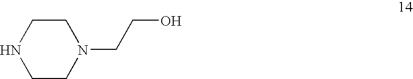

in the presence of a base and under microwave irradiation conditions to give compound of formula II

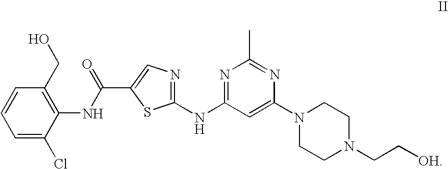

The compound 6 used in step d) is provided by a process comprising of
1) reacting a compound of formula 15

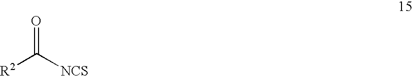

where R² is selected from alkyl and aryl, with a N-protected amine of formula 16

PG-NH₂ 16

where PG is as defined above, to give a compound of formula 17

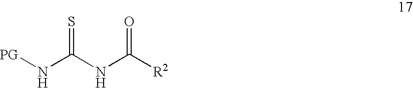

where R² and PG are as defined above;
2) reacting the compound of formula 17 with a base to give the compound of formula 18

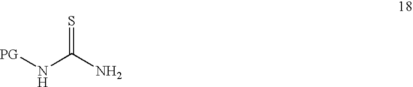

where PG is as defined above;
3) and reacting the compound of formula 18 with a compound of formula 19

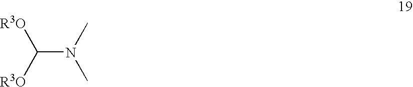

where R³ is alkyl, to give compound of formula 6

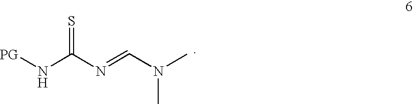

In yet another aspect, a process is provided for the preparation of the parent compound of formula I

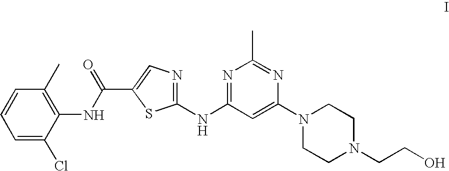

which includes the steps of
a) reacting the compound of formula 28

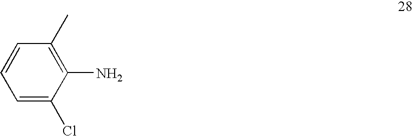

with a compound of formula 3

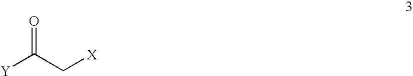

where X and Y are as defined above, in the presence of a base to give a compound of formula 29

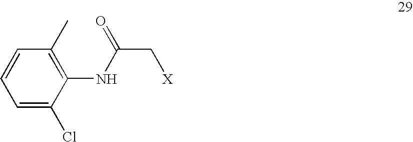

where X is as defined above;
b) reacting the compound of formula 23 with a compound of formula 6

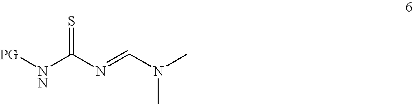

where PG is as defined above, to give a compound of formula 30

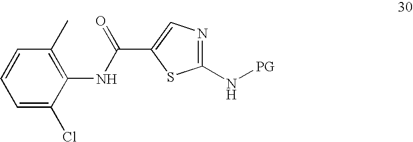

where PG is as defined above;
c) treating the compound of formula 24 with a deprotecting reagent to give a compound of formula 31

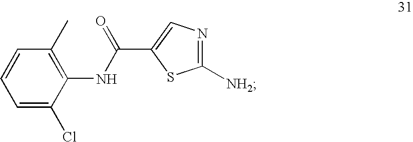

d) reacting the compound of formula 31 with a compound of formula 11

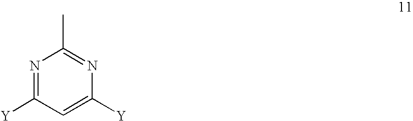

where X is as defined above, to give a compound of formula 32

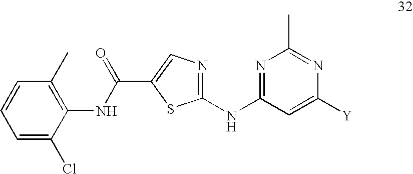

where X is as defined above;
e) and reacting the compound of formula 32 with a compound of formula 14

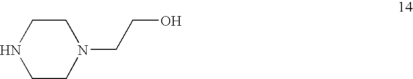

in the presence of a base and under microwave irradiation conditions to give compound of formula I

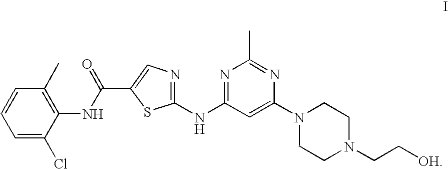

In still a further embodiment, a process is provided for preparing a compound of formula (IV)

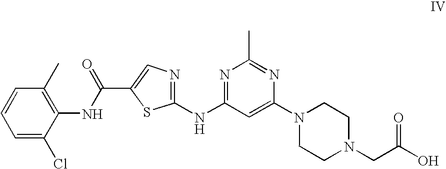

which includes the steps of
a) reacting the compound of formula 32

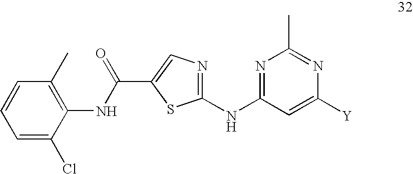

where X is as defined above, with a compound of formula 33

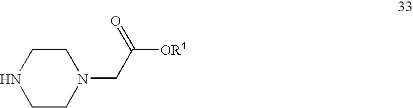

where R⁴ is selected from hydrogen, alkyl and aryl, under microwave irradiation conditions to give a compound of formula 34

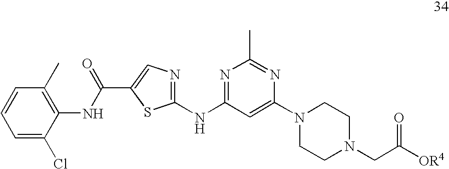

where R⁴ is as defined above;
b) treating the compound of formula 34 with a base to give compound of formula IV

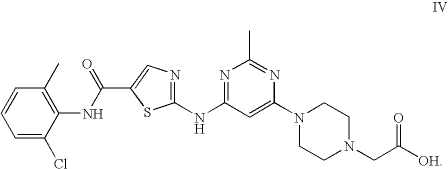

In still a further embodiment, a process is provided for preparing a compound of formula (IIa),

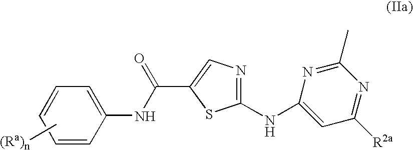

wherein

### R^{a} is selected from H, alkyl, halo, alkoxyl, C(O)OH, C(O)O-alkyl, alkyl-OC(O)—R¹, hydroxyalkyl, and/or suitable protecting groups thereof,

n is selected from 0, 1, 2 and 3;

### R¹ is selected from hydrogen, alkyl and aryl;

### R^{2a} is selected from halo,

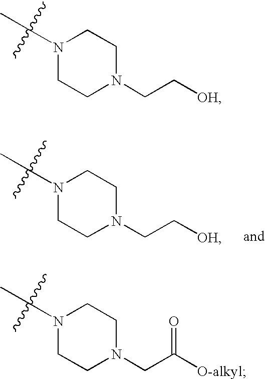

including the steps of reacting a compound of formula 6

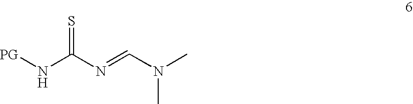

where PG is a nitrogen protecting group, with a compound of formula (A)

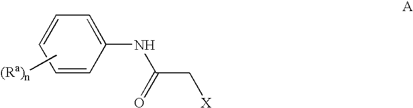

wherein X is selected from halo, mesylate, and tosylate;
to provide the compound of formula (Ia)

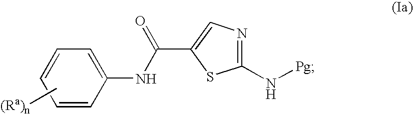

reacting the compound of formula Ia
with a deprotecting group to remove the Pg, followed by reaction with a compound of formula 2a

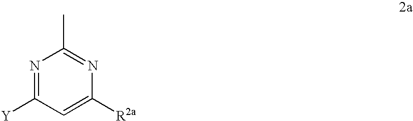

wherein Y is selected from Cl, Br, and I,
to give the compound of formula IIa.

In still a further embodiment, a process is provided for preparing the compound of formula IIa, wherein the compound of formula IIa has the formula (IIa′)

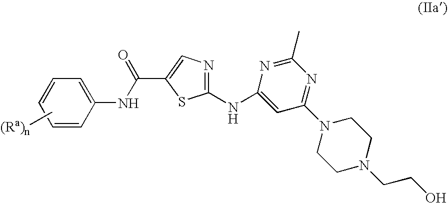

In still a further embodiment, a process is provided for preparing compounds, wherein

the compound of formula (A) is selected from

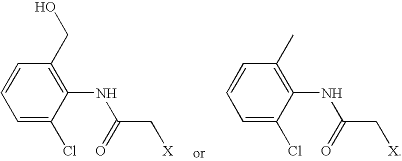

In still a further embodiment, a process is provided for preparing compounds, wherein

the compound of formula IIa has the formula II;

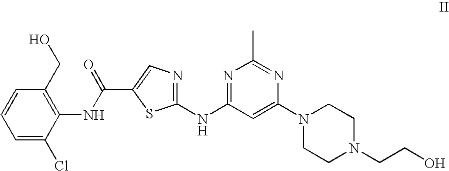

the process including the steps of
a) reacting the compound of formula 4 with a base to give a compound of formula 5

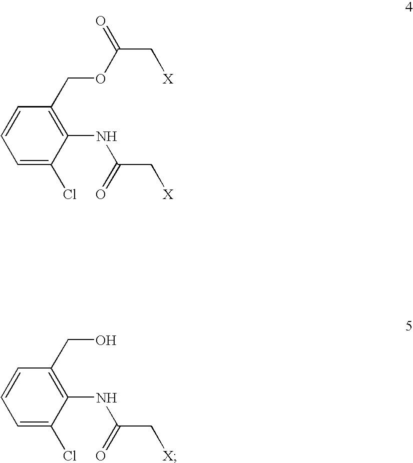

b) reacting the compound of formula 5 with a compound of formula 6

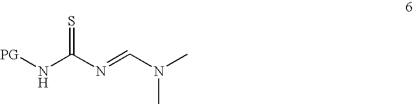

to give a compound of formula 7

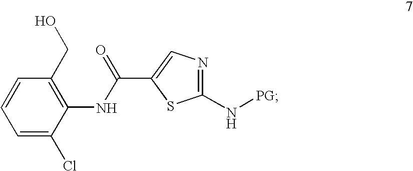

c) reacting the compound of formula 7 with a compound of formula 8

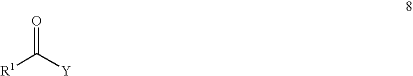

where Y is selected from Cl, Br, and I, and R¹ is selected from hydrogen, alkyl and aryl, to give a compound of formula 9

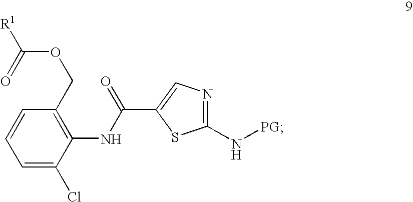

d) treating the compound of formula 9 with a deprotecting reagent to give a compound of formula 10

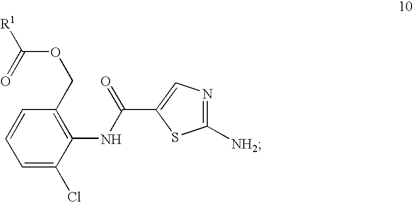

e) reacting the compound of formula 10 with a compound of formula 11

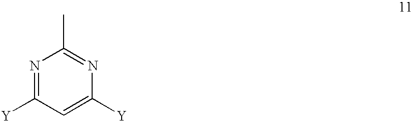

wherein Y is selected from Cl, Br, and I,
in the presence of a base to give compound of formula 12

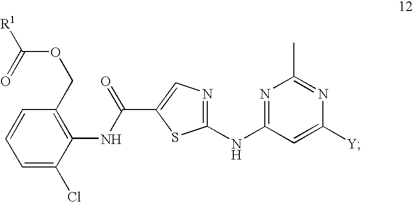

f) reacting the compound of formula 12 with a base to give compound of formula 13

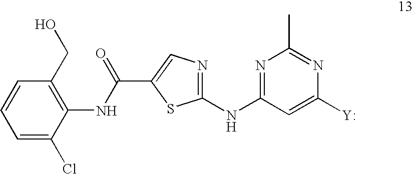

g) and reacting the compound of formula 13 with a compound of formula 14

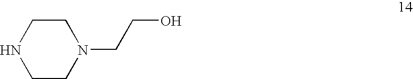

in the presence of a base and to give compound of formula II

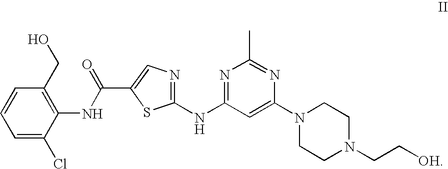

In still a further embodiment, a process is provided for preparing compounds, wherein

the compound formula IIa has the formula I;

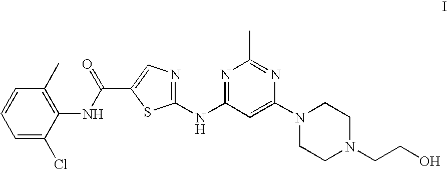

including the steps of
a) reacting the compound of formula 29 with a compound of formula 6

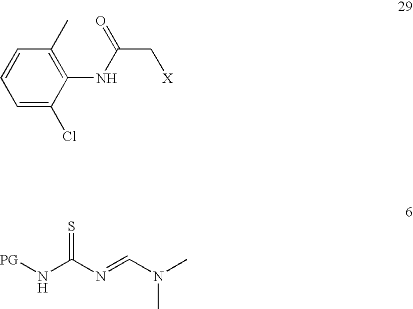

to give a compound of formula 30;

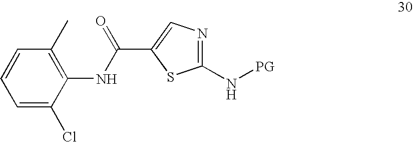

c) treating the compound of formula 30 with a deprotecting reagent to give a compound of formula 31

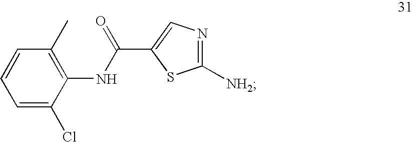

d) reacting the compound of formula 31 with a compound of formula 11

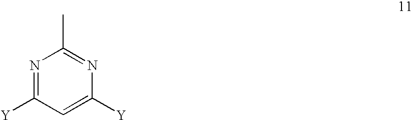

to give a compound of formula 32

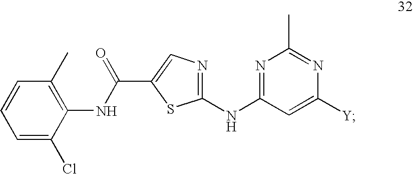

e) and reacting the compound of formula 32 with a compound of formula 14

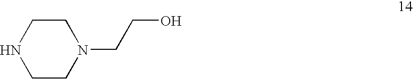

in the presence of a base conditions to give compound of formula I

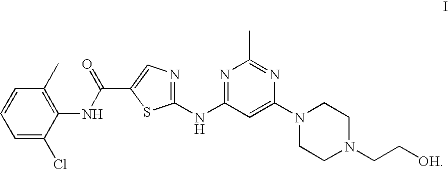

In still a further embodiment, a process is provided for preparing compounds, wherein

the compound of formula IIa has the formula II;

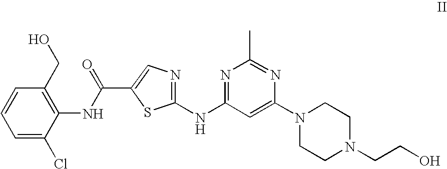

the process includes the steps of
a) reacting a compound with a formula 1

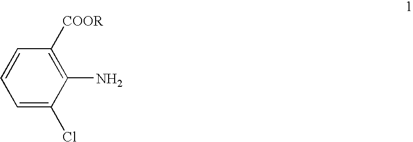

where R is selected from hydrogen, alkyl and aryl, with a reducing reagent to give a compound of formula 2

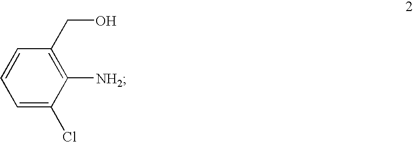

b) reacting the compound of formula 2 with a compound of formula 3

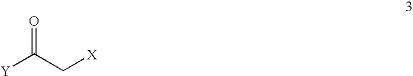

wherein Y is selected from Cl, Br, and I, in the presence of a base to give a compound of formula 4

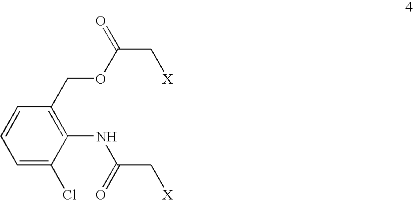

where X is as defined above;
c) reacting the compound of formula 4 with a base to give a compound of formula 5;

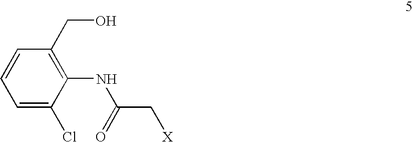

d) reacting the compound of formula 5 with a compound of formula 6

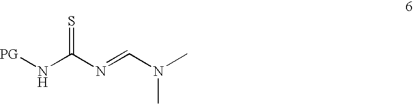

to give a compound of formula 7;

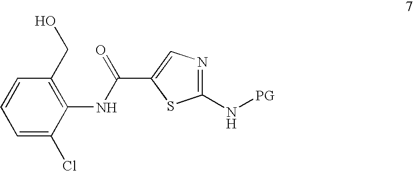

e) reacting the compound of formula 7 with a compound of formula 8

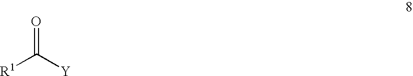

and R¹ is selected from hydrogen, alkyl and aryl, to give a compound of formula 9;

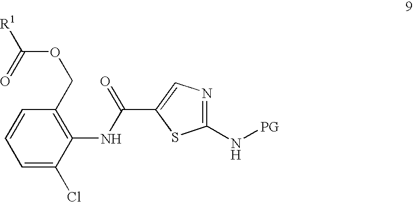

g) treating the compound of formula 9 with a deprotecting reagent to give a compound of formula 10;

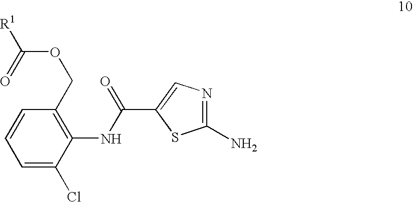

h) reacting the compound of formula 10 with a compound of formula 11

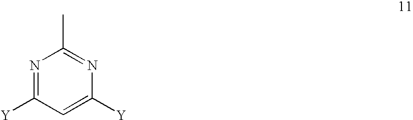

in the presence of a base to give compound of formula 12;

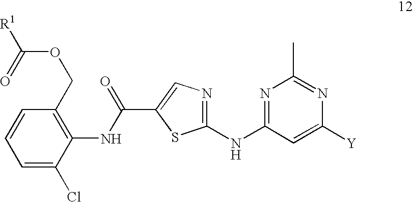

i) reacting the compound of formula 12 with a base to give compound of formula 13;

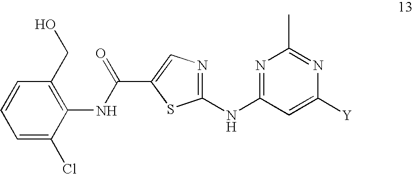

j) and reacting the compound of formula 13 with a compound of formula 14

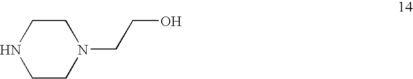

in the presence of a base and to give compound of formula II

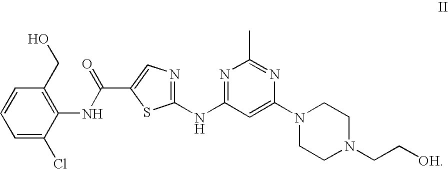

In still a further embodiment, a process is provided for preparing compounds, wherein

the compound formula IIa has the formula I;

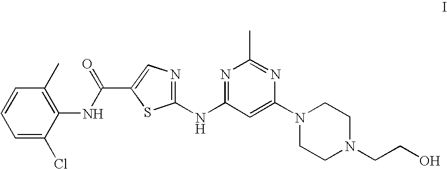

including the steps of
a) reacting the compound of formula 28

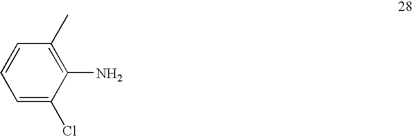

with a compound of formula 3

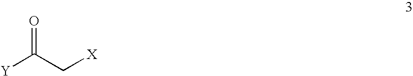

wherein Y is selected from Cl, Br, and I, in the presence of a base to give a compound of formula 29

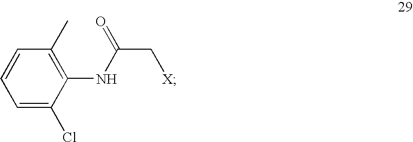

b) reacting the compound of formula 29 with a compound of formula 6

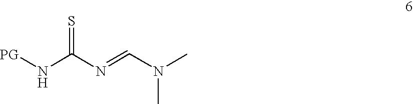

to give a compound of formula 30;

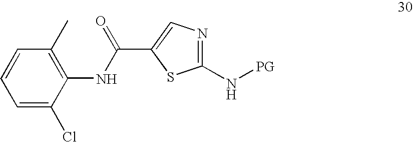

c) treating the compound of formula 30 with a deprotecting reagent to give a compound of formula 31

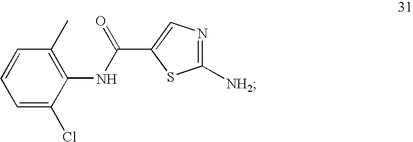

d) reacting the compound of formula 31 with a compound of formula 11

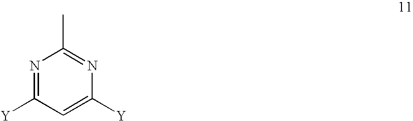

to give a compound of formula 32

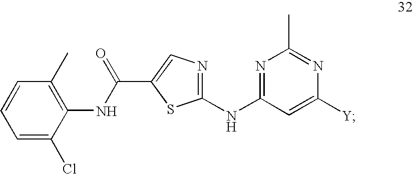

e) and reacting the compound of formula 32 with a compound of formula 14

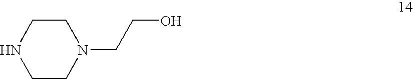

in the presence of a base to give compound of formula I

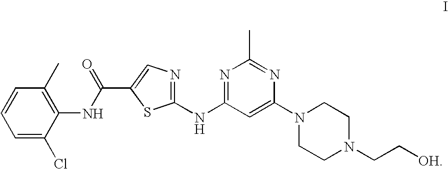

In still a further embodiment, a process is provided for preparing compounds, wherein the compound of formula IIa has the formula IV

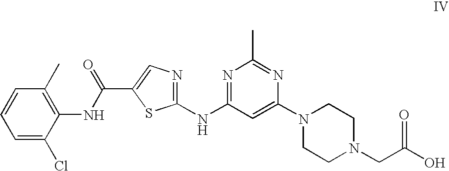

which includes the steps of
a) reacting the compound of formula 32

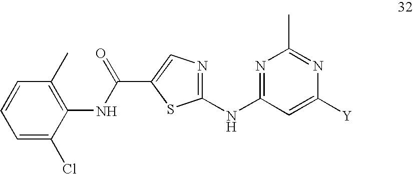

where Y is selected from Cl, Br, and I, with a compound of formula 33

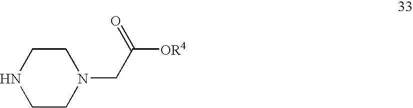

where R⁴ is selected from hydrogen, alkyl and aryl, to give a compound of formula 34

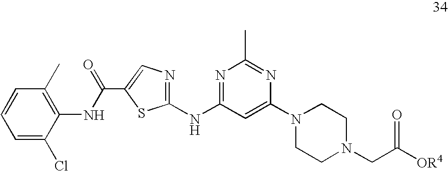

where R⁴ is as defined above;
b) treating the compound of formula 34 with a base to give compound of formula IV

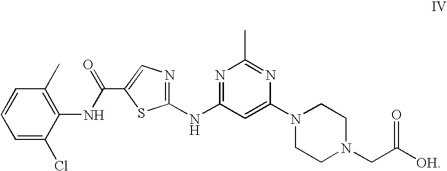

In still a further embodiment, a process is provided for preparing compounds, wherein

### Pg is selected from benzyl, p-methoxybenzyl, diphenylmethyl and trityl; and

the step of reacting the compound of formula 6 with the compound of formula (A) is done in a solvent selected from MeOH, EtOH, and Pr—OH.

In still a further embodiment, a process is provided for preparing compounds, wherein R^{a} is selected from hydrogen alkyl, halo, hydroxyalkyl, C(O)OH, C(O)O-alkyl, alkyl-OC(O)—R¹.

In still a further embodiment, a process is provided for preparing compounds, wherein

### Pg is selected from benzyl, p-methoxybenzyl, diphenylmethyl and trityl; and

the step of reacting the compound of formula 6 with the compound of formula (A) is done in a solvent selected from MeOH, EtOH, and Pr—OH.

The invention may be embodied in other specific forms without departing from the spirit or essential atributes thereof. This invention also encompasses all combinations of alternative aspects of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### Definitions

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds. The structures of the metabolites may be shown with a substituent bonded to a portion of the molecule. This is meant to indicate that that substituent may be present at any location on that portion of the molecule and the exact location is not definitely known.

The compounds may form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. In addition, when a compound of formula I contains both a basic moiety, such as, but not limited to an amine or a pyridine ring, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound of the formula I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

The compounds disclosed herein, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention. Additionally, inventive compounds may have trans and cis isomers and may contain one or more chiral centers, therefore existing in enantiomeric and diastereomeric forms. The invention includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Substantially pure” as used herein is intended to include a compound having a purity greater than 90 weight percent, including 90, 91, 92, 93, 94, 95, 96, 97, 98, 99, and 100 percent.

The term “alkyl” as used herein by itself or as part of another group refers to straight and branched chain saturated hydrocarbons, containing 1 to 8 carbons, such as methyl, ethyl, propyl, isopropyl, butyl, t-butyl, isobutyl, pentyl, hexyl, isohexyl, heptyl, 4,4-dimethylpentyl, octyl, 2,2,4-trimethyl-pentyl, nonyl, decyl, undecyl, dodecyl, the various branched chain isomers thereof, and the like. In another embodiment, alkyl is of 1 to 4 carbon atoms.

The terms “ar” or “aryl” as used herein by itself or as part of another group refer to optionally-substituted aromatic homocyclic (i.e., hydrocarbon) monocyclic, bicyclic or tricyclic aromatic groups containing 6 to 14 carbons in the ring portion [such as phenyl, biphenyl, naphthyl (including 1-naphthyl and 2-naphthyl) and antracenyl], and may optionally include one to three additional rings (either cycloalkyl, heterocyclo or heteroaryl) fused thereto. Examples include:

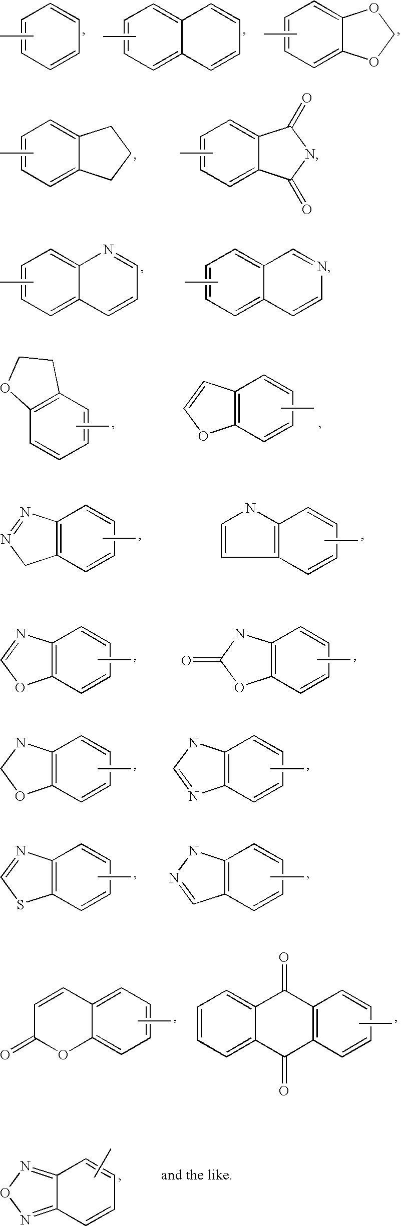

The term “halo” or “halogen” refers to chloro, bromo, fluoro and iodo, alternatively, chloro or bromo.

The term “protecting group” as used herein refers to any group known in the art of organic synthesis for the protection of functional groups. As used herein, the term “amine protecting group” or “nitrogen protecting group” refers to any group known in the art of organic synthesis for the protection of amine groups. As used herein, the term “amine protecting group reagent” refers to any reagent known in the art of organic synthesis for the protection of amine groups which may be reacted with an amine to provide an amine protected with an amine protecting group. The “amine protecting group” should be compatible with other reaction conditions. Such amine protecting groups include those listed in Greene and Wuts, “Protective Groups in Organic Synthesis” John Wiley & Sons, New York (1991) and “The Peptides: Analysis, Synthesis, Biology, Vol. 3, Academic Press, New York (1981), the disclosure of which is hereby incorporated by reference. Examples of amine protecting groups include, but are not limited to, the following: 1) acyl types such as formyl, trifluoroacetyl, and p-toluenesulfonyl; 2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; and 4) cyclic alkyl carbamate types such as cyclopentyloxycarbonyl and adamantyloxycarbonyl; and 5) benzyl and substituted benzyl groups.

Amine protecting groups may include, but are not limited to the following: benzyl, p-methoxybenzyl, diphenylmethyl or trityl, 2,7-di-t-butyl-[9-(10,10-dioxo-10,10,10,10-tetrahydrothio-xanthyl)]methyloxycarbonyl; 2-trimethylsilylethyloxycarbonyl; 2-phenylethyloxycarbonyl; 11-dimethyl-2,2-dibromoethyloxycarbonyl; 1-methyl-1-(4-biphenylyl)ethyloxycarbonyl; benzyloxycarbonyl; p-nitrobenzyloxycarbonyl; 2-(p-toluenesulfonyl)ethyloxycarbonyl; m-chloro-p-acyloxybenzyloxycarbonyl; 5-benzyisoxazolylmethyloxycarbonyl; p-(dihydroxyboryl)benzyloxycarbonyl; m-nitrophenyloxycarbonyl; o-nitrobenzyloxycarbonyl; 3,5-dimethoxybenzyloxycarbonyl; 3,4-dimethoxy-6-nitrobenzyloxycarbonyl; N′-p-toluenesulfonylaminocarbonyl; t-amyloxycarbonyl; p-decyloxybenzyloxycarbonyl; diisopropylmethyloxycarbonyl; 2,2-dimethoxycarbonylvinyloxycarbonyl; di(2-pyridyl)methyloxycarbonyl; or 2-furanylmethyloxycarbonyl.

“Suitable solvent” as used herein is intended to refer to a single solvent as well as mixtures of solvents. Solvents may be selected, as appropriate for a given reaction step, from, for example, aprotic polar solvents such as DMF, DMA, DMSO, dimethylpropyleneurea, N-methylpyrrolidone (NMP), and hexamethylphosphoric triamide; ether solvents such as diethyl ether, THF, 1,4-dioxane, methyl t-butyl ether, dimethoxymethane, and ethylene glycol dimethyl ether; alcohol solvents such as MeOH, EtOH, and isopropanol; and halogen-containing solvents such as methylene chloride, chloroform, carbon tetrachloride, and 1,2-dichloroethane. Mixtures of solvents may also include biphasic mixtures.

“Reducing agent” or “reducing reagent” as used herein is a reagent or combination of reagents which will selectively reduce the selected functional group. The selection of the individual reducing agent will depend on the specific functional group being reduced and other features of the compound on which the reaction is taking place. Examples of reducing agents include, but are not limited to, agents such as such as LAH, diborane, and/or LiBH₄,

“Base” includes both organic and inorganic bases. The strength of the individual base will depend on the specific reaction. Examples of bases include, but are not limited to, TEA (triethylamine), NMM (N-methyl morpholine), pyridine, NaH, NaOBu-t, KOBu-t, ethyldiisopropylamine, NaOH, KOH and/or LiOH.

“Deprotecting Agent” as used herein is a reagent or combination of reagents which will selectively remove the protecting group. The selection of the deprotecting agent to be used in a specific reaction will depend the protecting group and other features of the compound on which the reaction is taking place. Examples of deprotecting agents include, but are not limited to, agents such as HCl, HCO₂H, and/or TFA.

“Chlorinating Agent” as used herein is a reagent or combination of reagents which will selecting add a chloro substitutent to a molecule of interest. Examples of chlorinating agents include, but are not limited to, agents such as Cl₂, NCS and/or HCl/H₂O.

The compounds of formula I-IV can be prepared by processes of the invention as shown in the following reaction schemes and description thereof Exemplary reagents and procedures for these reactions appear hereinafter and in the working Examples.

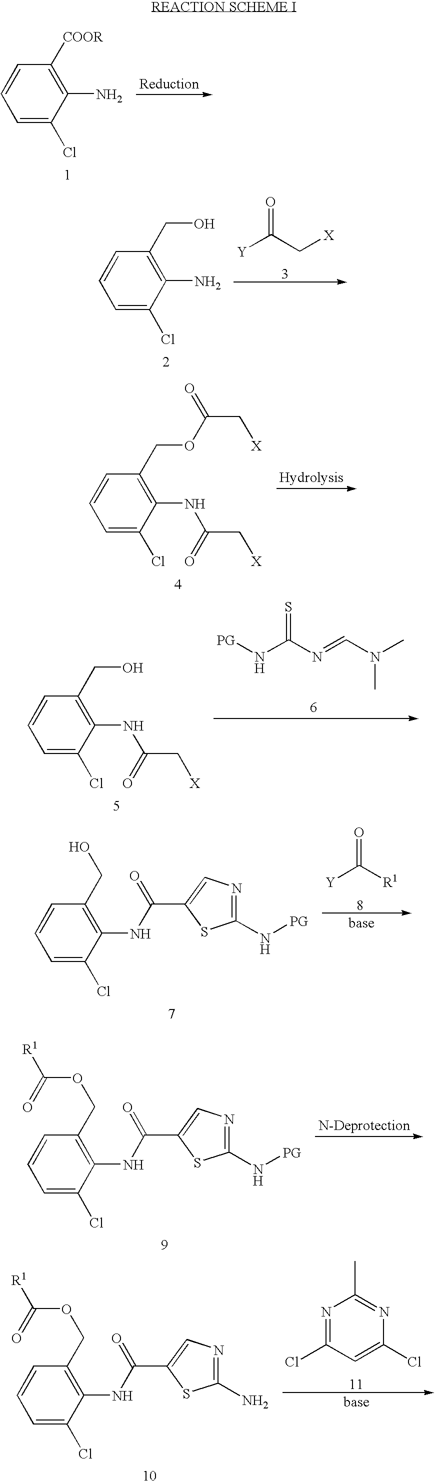

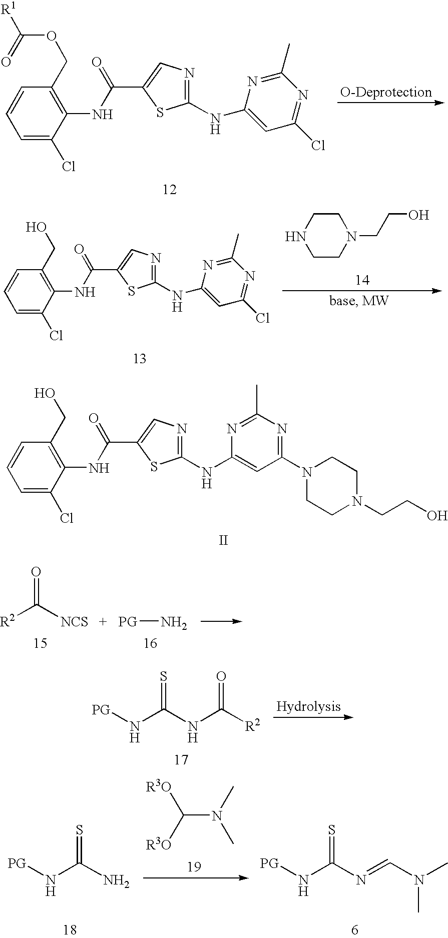

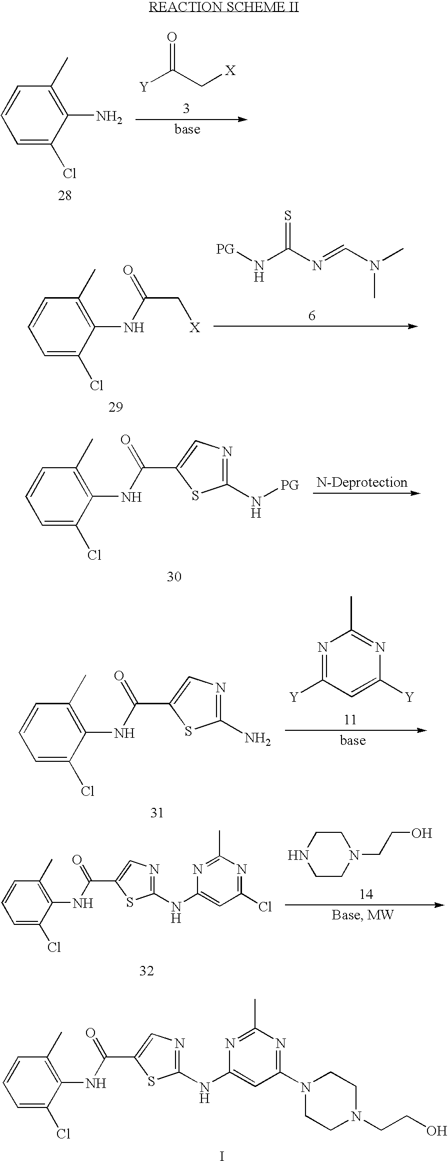

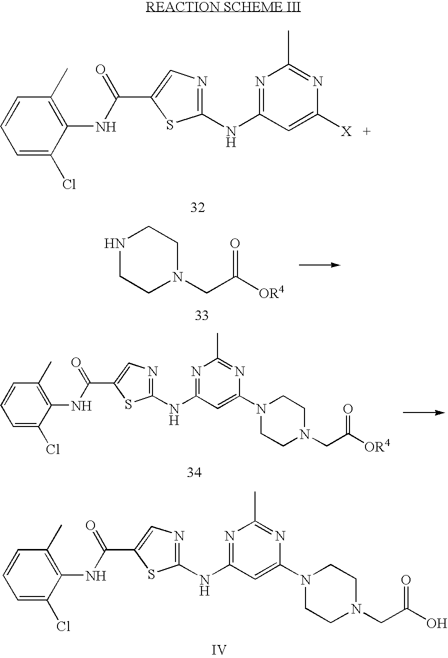

Reaction Scheme I sets out a process for preparing the compound of formula II

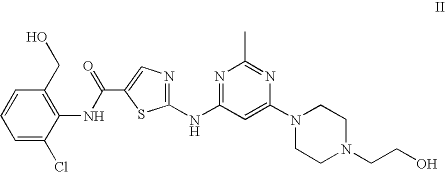

wherein a compound with a formula 1

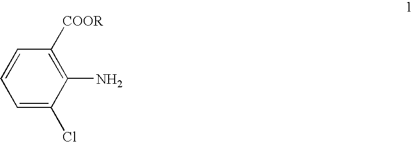

wherein R is as defined above and is hydrogen, alkyl or aryl, alternatively, hydrogen, in a suitable organic solvent, such as ether, THF, or dioxane, alternatively THF, is treated with a reducing reagent, such as LAH, diborane, or LiBH₄, alternatively LAH to give a compound of formula 2

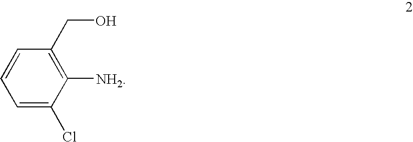

Compound of formula 2 is then made to undergo a reaction with a compound of formula 3

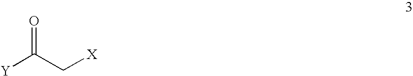

where X is halo, mesylate, or tosylate, alternatively, Cl, Br, or I, and Y is as defined above and is preferably Cl, Br, or I, alternatively Cl in the presence of a base, such as pyridine, triethylamine, diisopropylethylamine, alternatively pyridine, in an organic solvent such as dichloromethane, chloroform or dichloroethane, alternatively dichloromethane to give a compound of formula 4

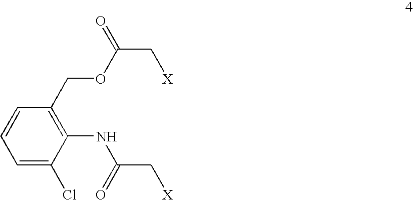

where X is as defined above.

Compound of formula 4 is hydrolyzed with a base such as LiOH, NaOH, or KOH, alternatively LiOH, in an aqueous solvent such as MeOH, EtOH or PrOH, alternatively MeOH, to give a compound of formula 5

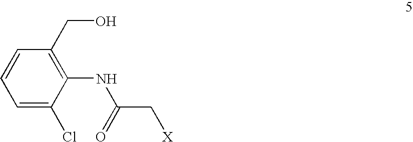

where X is as defined above.

Compound of formula 5 is made to undergo a cyclization reaction with a compound of formula 6

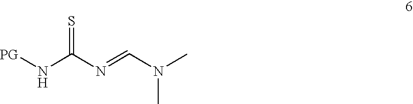

where PG is a nitrogen protecting group, such as benzyl, p-methoxybenzyl, diphenylmethyl or trityl, alternatively trityl, in an organic solvent such as MeOH, EtOH, or Pr—OH (propanol), alternatively MeOH to give a compound of formula 7

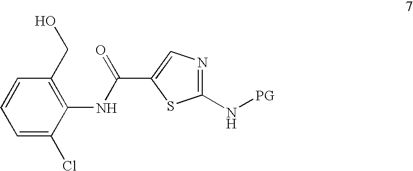

where PG is as defined above.

The compound of formula 7 is made to undergo a reaction with a compound of formula 8

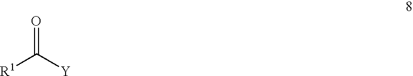

where Y is as defined above and R¹ is hydrogen, alkyl or aryl, alternatively alkyl, alternatively t-butyl in an organic solvent such as THF (tetrahydrofuran), ether, dichloromethane or ethyl acetate, alternatively THF, in the presence of a base such as TEA (triethyl amine), NMM (N-methylmorpholine) or pyridine, alternatively NMM, to give a compound of formula 9

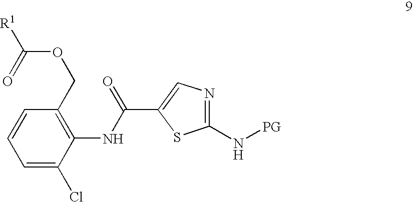

where R¹ and PG are as defined above.

The compound of formula 9 is treated with a deprotecting reagent such as HCl, HCO₂H, or TFA (trifluoroacetic acid), alternatively HCO₂H in an organic solvent such as MeOH, EtOH or PrOH, alternatively EtOH, to give a compound of formula 10

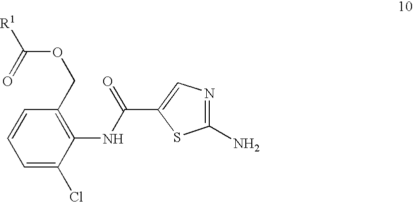

where R¹ is as defined above.

The compound of formula 10 is made to react with a compound of formula 11

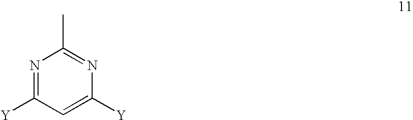

where X is as defined above, in an organic solvent such as THF, Ether or dioxane, alternatively THF, in the presence of a base such as NaH, NaOBu-t or KOBu-t, alternatively NaOBu-t, to give compound of formula 12

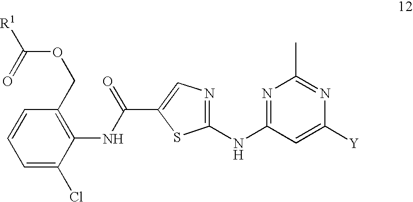

where X is as defined above.

The compound of formula 12 is treated with a base such as LiOH, NaOH or KOH, in the presence of an aqueous solvent such as THF, dioxane or MeOH, alternatively THF, to give compound of formula 13

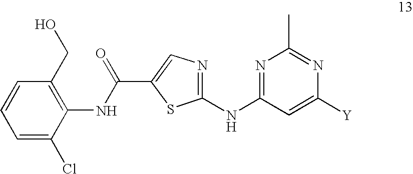

where X is as defined above.

The compound of formula 13 is made to undergo reaction with a compound of formula 14

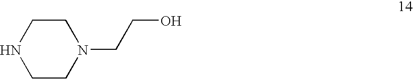

in the presence of a base such as ethyldisiopropylamine, TEA or NMM, alternatively ethyldiisopropylamine, in an organic solvent such as MeOH, EtOH or PrOH, alternatively EtOH, to give compound of formula II Alternatively, the reaction may be run under microwave irradiation conditions to give compound of formula II

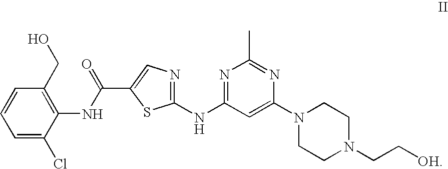

The compound 6 used in the above process is provided by reacting a compound of formula 15

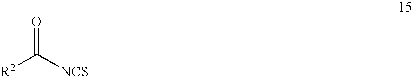

where R² is alkyl or aryl, alternatively aryl, alternatively phenyl, in the presence of an organic solvent such as chloroform, dichloromethane or toluene, alternatively chloroform, with a N-protected amine of formula 16

PG-NH₂ 16

where PG is as defined above, to give a compound of formula 17

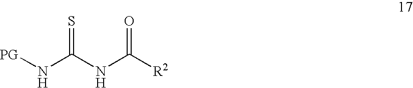

where R² and PG are as defined above.

The compound of formula 17 is next made to undergo a reaction with a base such as NaOH, LiOH or KOH, alternatively NaOH, in an aqueous solvent such as MeOH, EtOH or PrOH, to give the compound of formula 18

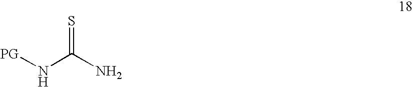

where PG is as defined above.

The compound of formula 18 is then made to undergo reaction with a compound of formula 19

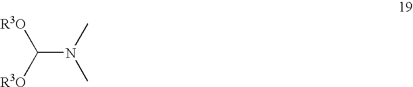

where R³ is alkyl such as Me, Et or Bu, alternatively Me, in an organic solvent such as MeOH, EtOH or BuOH, alternatively MeOH, to give compound of formula 6

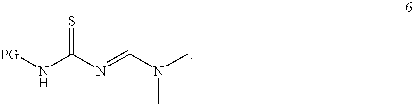

Reaction Scheme II sets out a process for preparing the parent compound of formula I

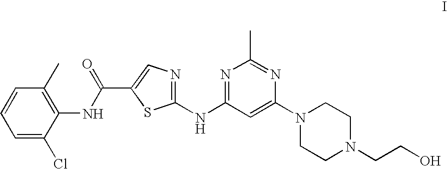

wherein the compound of formula 28

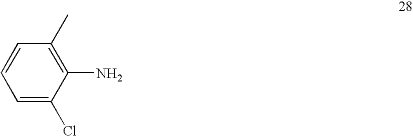

with a compound of formula 3

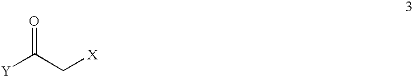

where X and Y are as defined above, in an organic solvent such as dichloromethane, dichloroethane or THF, alternatively dichloromethane, in the presence of a base such as pyridine, TEA or NMM, alternatively pyridine, to give a compound of formula 29

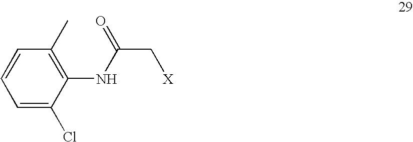

where X is as defined above.

The compound of formula 23 is made to undergo a reaction with a compound of formula 6

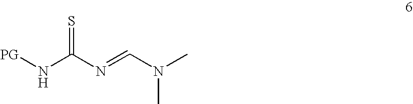

where PG is as defined above, in an organic solvent such as MeOH, EtOH or PrOH, alternatively EtOH, to give a compound of formula 30

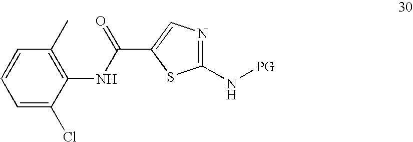

where PG is as defined above.

The compound of formula 24 is treated with a deprotecting reagent such as HCl, HCO₂H, or TFA, alternatively HCO₂H in an organic solvent such as MeOH, EtOH or PrOH, alternatively EtOH, to give a compound of formula 31

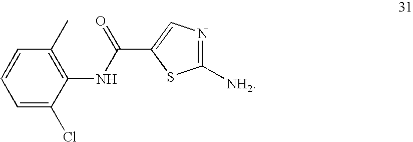

The compound of formula 31 is reacted with a compound of formula 11

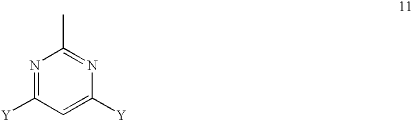

where X is as defined above, in an organic solvent such as THF, Ether or dioxane, alternatively THF, in the presence of a base such as NaH, NaOBu-t or KOBu-t, alternatively NaOBu-t, to give a compound of formula 32

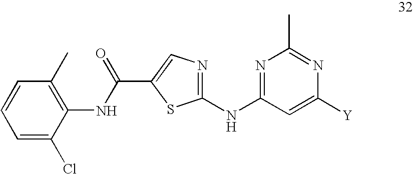

where X is as defined above.

The compound of formula 32 is treated with a compound of formula 14

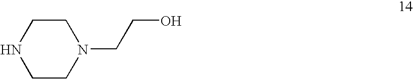

in the presence of a base such as ethyldisiopropylamine, TEA or NMM, alternatively ethyldiisopropylamine, in an organic solvent such as MeOH, EtOH or PrOH, alternatively EtOH, to give compound of formula I

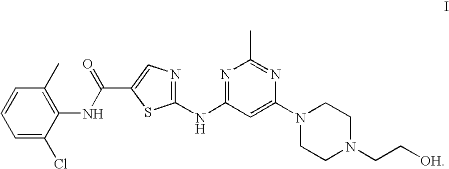

Reaction Scheme III sets out a process for preparing a compound of formula IV

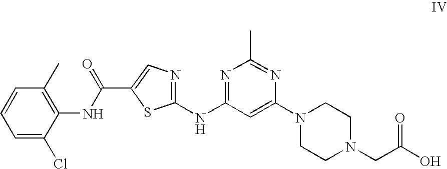

wherein the compound of formula 32

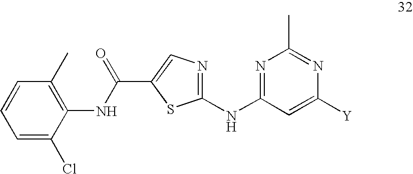

where X is as defined above, is treated with a compound of formula 33

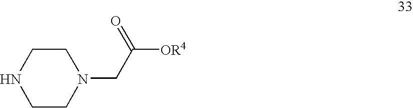

where R⁴ is hydrogen, alkyl or aryl, preferably alkyl, more preferably ethyl, in an organic solvent such as MeOH, EtOH or PrOH, alternatively EtOH, to give a compound of formula 34. Alternatively the reaction may be run under microwave irradiation conditions, to give a compound of formula 34

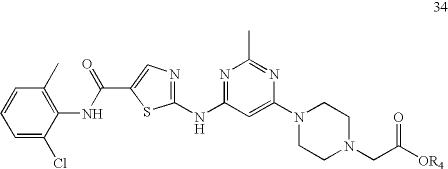

where R⁴ is as defined above.

The compound of formula 34 is next treated with a base such as NaOH, KOH or LiOH, alternatively NaOH, in an organic solvent such as MeOH, EtOH or PrOH, alternatively MeOH, to give compound of formula IV

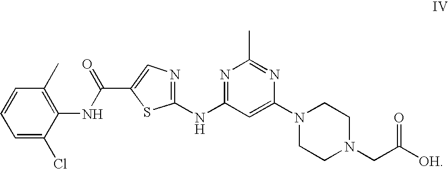

### Abbreviations

The following abbreviations are employed herein and/or in the following Examples:
AIBN=2,2′-azobisisobutyronitrile (AIBN)
DCC=1,3-dicyclohexylcarbodiimide
CDl=carbonyl diimidazole
Ph=phenyl
Bn=benzyl
t-Bu=tertiary butyl
Me=methyl
Et=ethyl
TMS=trimethylsilyl
TMSN₃=trimethylsilyl azide
ITBS=tert-butyldimethylsilyl
FMOC=fluorenylmethoxycarbonyl
Boc=tert-butoxycarbonyl
Cbz=carbobenzyloxy or carbobenzoxy or benzyloxycarbonyl
THF=tetrahydrofuran
Et₂O=diethyl ether
hex=hexanes
EtOAc=ethyl acetate
DMF=dimethyl formamide
MeOH=methanol
EtOH=ethanol
i-PrOH=isopropanol
DMSO=dimethyl sulfoxide
DME=1,2 dimethoxyethane
DCE=1,2 dichloroethane
HOAc or AcOH=acetic acid
TFA=trifluoroacetic acid
i-Pr₂NEt=diisopropylethylamine
Et₃N=triethylamine
NMM=N-methyl morpholine
DMAP=4-dimethylaminopyridine
NaBH₄=sodium borohydride
NaBH(OAc)₃=sodium triacetoxyborohydride
DIBALH=diisobutyl aluminum hydride
LiAlH₄=lithium aluminum hydride
n-BuLi=n-butyllithium
Pd/C=palladium on carbon
PtO₂=platinum oxide
KOH=potassium hydroxide
NaOH=sodium hydroxide
LiOH=lithium hydroxide
K₂CO₃=potassium carbonate
NaHCO₃=sodium bicarbonate
DBU=1,8-diazabicyclo[5.4.0]undec-7-ene
EDC (or EDC.HCl) or EDCI (or EDCI.HCl) or EDAC=3-ethyl-3-(dimethylamino)propyl-carbodiimide hydrochloride (or 1-(3-dimethylaminopropyl)-3-ethylcarbodiimide hydrochloride)
HOBT or HOBT.H₂O=1-hydroxybenzotriazole hydrate
HOAT=1-Hydroxy-7-azabenzotriazole
BOP reagent=benzotriazol-1-yloxy-tris (dimethylamino) phosphonium hexafluorophosphate
NaN(TMS)₂=sodium hexamethyldisilazide or sodium bis(trimethylsilyl)amide
Ph₃P=triphenylphosphine
Pd(OAc)₂=Palladium acetate
(Ph₃P)₄Pd°=tetrakis triphenylphosphine palladium
DEAD=diethyl azodicarboxylate
DIAD=diisopropyl azodicarboxylate
Cbz-Cl=benzyl chloroformate
CAN=ceric ammonium nitrate
SAX=Strong Anion Exchanger
SCX=Strong Cation Exchanger
Ar=argon
N₂=nitrogen
min=minute(s)
h or hr=hour(s)
L=liter
mL=milliliter
μL=microliter
g=gram(s)
mg=milligram(s)
mol=moles
mmol=millimole(s)
meq=milliequivalent
RT=room temperature
sat or sat'd=saturated
aq.=aqueous
TLC=thin layer chromatography
HPLC=high performance liquid chromatography
LC/MS=high performance liquid chromatography/mass spectrometry
MS or Mass Spec=mass spectrometry
NMR=nuclear magnetic resonance
NMR spectral data: s=singlet; d=doublet; m=multiplet; br=broad; t=triplet
mp=melting point

### EXAMPLES

The following examples illustrate the invention but should not be interpreted as a limitation thereon.

### Preparation of N,N-dimethyl-N′-(tritylcarbamothioyl)formimidamide (6a)

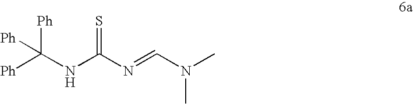

The title compound was prepared according to the following reaction scheme:

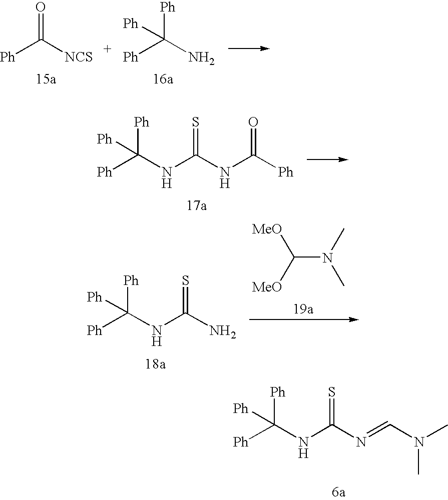

### A. Preparation of N-(tritylcarbamothioyl)benzamide (17a)

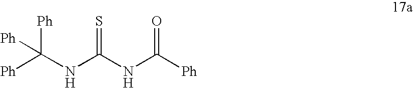

In a 5 L round bottomed flask equipped with an overhead stirrer and an N₂ inlet were introduced tritylamine (16a) (159.0 g, 613 mmol) and chloroform (1.6 L). The mixture was cooled to −2° C., and then benzoyl isothiocyanate (15a, 82.6 mL, 613 mmol) was added in a slow stream at 0-5° C. The mixture was stirred at 0-5° C. for 45 mins and then was allowed to warm to room temperature over a period of 3.5 h. The mixture was concentrated in vacuo to give the crude compound 17a as a yellow solid which was stored at 3° C. overnight which was used as is in the next step.

### B. Preparation of 1-tritylthiourea (18a)

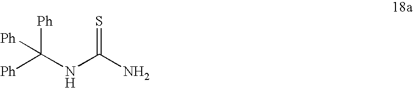

The yellow solid 17a obtained in the previous step was suspended in a solution of NaOH (248 g, 7.1 mol) in MeOH (1.6 L) and H₂O (223 mL). The suspension was heated to 60° C. and then stirred for 2 h. The resulting suspension was then concentrated to a white solid, which was triturated with H₂O (1.5 L) at room temperature for 35 mins and vacuum filtered. The filter cake was rinsed with H₂O and vacuum air dried to give compound 18a as a white solid (193.8 g, 99% yield for two steps). ¹H NMR (300 MHz, DMSO-d₆) δ 7.25-7.35 (m, 15H).

### C. Preparation of N,N-dimethyl-N′-(tritylcarbamothioyl)formimidamide (6a)

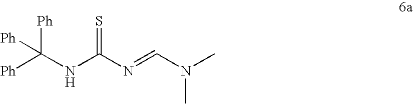

In a 5 L round bottomed flask equipped with an overhead stirrer and an N₂ inlet were introduced compound 18a (150.0 g, 471 mmol), MeOH (3 L) and dimethylformamide dimethyl acetal (19a, 80.0 mL, 600 mmol). The mixture was stirred at reflux for 2 h and then was cooled to room temperature. The resulting suspension was vacuum filtered. The filter cake was rinsed with MeOH and dried to give 1-trityl-3-((dimethylamino)methylene)thiourea (6a) as a white solid (157.9 g, 90% yield). 1H NMR (300 MHz, DMSO-d6) δ 2.27 (S, 3H), 2.95 (s, 3H), 7.17-7.32 (m 15H), 8.17 (s, 1H), 8.25 (s, 1H).

### Example 1

### Preparation of N-(2-chloro-6-(hydroxymethyl)phenyl)-2-(6-(4-(2-hydroxyethyl)piperazin-1-yl)-2-methylpyrimidin-4-ylamino)thiazole-5-carboxamide (II)

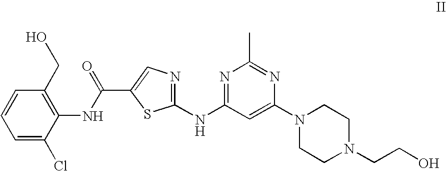

The title compound was prepared according to the following reaction scheme:

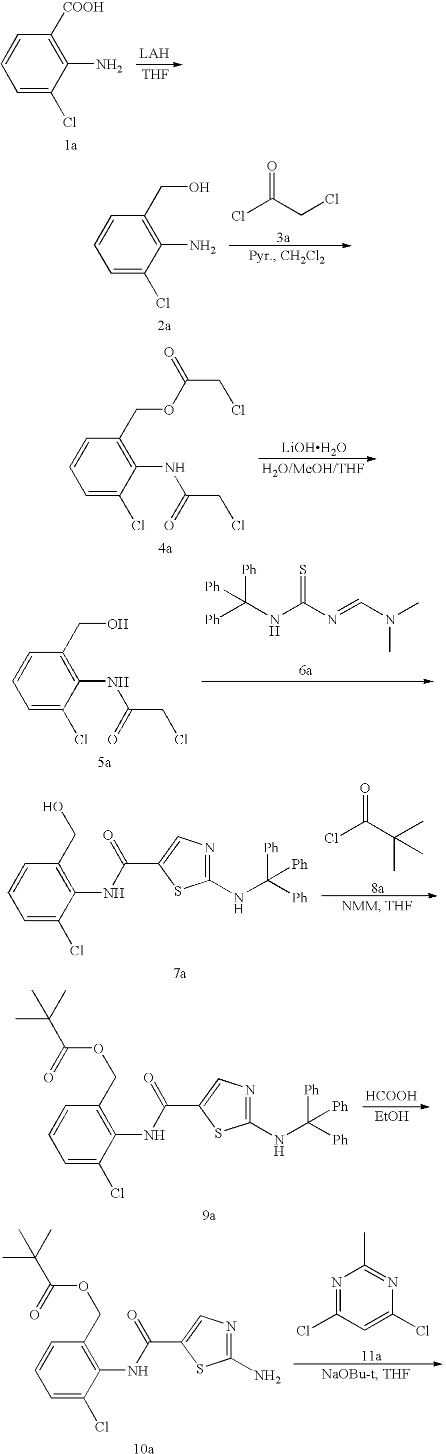

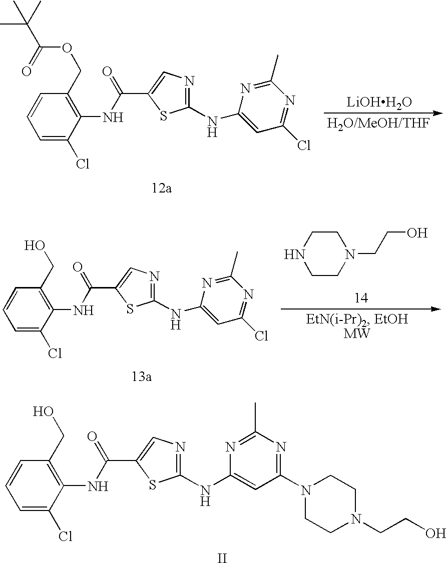

### A. Preparation of compound (2-amino-3-chlorophenyl)methanol (2a):

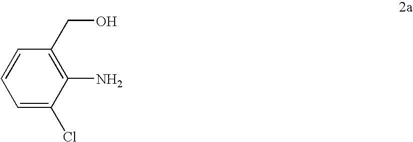

To a solution of lithium aluminum hydride in THF (210 mL, 1 M, 210 mmol) at room temperature, under N₂, was added slowly a solution of compound 1a (15.0 g, 87.4 mmol) in THF (200 mL) over a period of 20 mins. The reaction mixture was stirred for another 1.5 h at room temperature before it was cooled to 10° C. Water (300 mL) was added slowly. The resulting suspension was filtered through a celite pad. The celite pad was rinsed with EtOAc (1 L). The filtrate was washed with brine (3×300 mL), dried over Na₂SO₄, filtered and concentrated in vacuo to give the product 2a (13.0 g, 94% yield). ¹H NMR (500 MHz, CDCl₃) δ 4.69 (s, J=5.0 Hz, 1H), 6.63 (t, J=5.2 Hz, 1H), 6.97 (d, J=5.0, 1H), 7.24 (d, J=5.0 Hz, 1H).

### B. Preparation of compound 3-chloro-2-(2-chloroacetamido)benzyl 2-chloroacetate (4a)

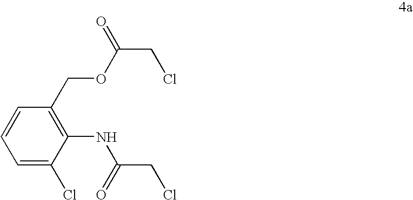

Compound 2a (13.0 g, 82.5 mmol) was dissolved in CH₂Cl₂ (500 mL). The solution was cooled to −10° C. Pyridine (20 mL) was added followed by addition of chloroacetyl chloride (16.4 mL, 206.5 mmol). The reaction mixture was allowed to warm to 10° C. over a period of 2.5 h. It was then quenched with 1 N HCl (500 mL). The organic phase was separated. The aqueous layer was extracted with CH₂Cl₂ (300 mL). The combined organic extracts were washed with H₂O (500 mL), brine (500 mL), dried over MgSO₄, filtered and concentrated in vacuo to give the crude product 3 which was recrystallized from 30% EtOAc in hexane to give the compound 4a (24.8 g, 97% yield). ¹H NMR (500 MHz, CDCl₃) δ 4.08 (s, 2H), 4.26 (s, 2H), 5.19 (s, 2H), 7.30 (t, J=5.0 Hz, 1H), 7.38 (d, J=5.0 Hz, 1H), 7.48 (d, J=5.0 Hz, 1H); ¹³C (125 MHz, CDCl₃) δ 40.73, 42.75, 64.62, 128.33, 128.87, 130.34, 131.81, 132.18, 134.53, 165.11, 167.09; Anal. Calcd for C₁₁H₁₀C₁₃NO₃: C, 42.54; H, 3.24; N, 4.51. Found: C, 42.63; H, 3.19; N, 4.29.

### C. Preparation of compound 2-chloro-N-(2-chloro-6-(hydroxymethyl)phenyl)acetamide (5a)

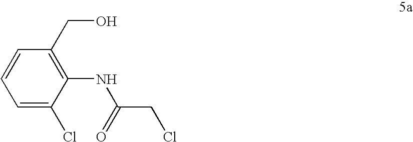

Lithium hydroxide mono hydrate (12.55 g, 306 mmol) was dissolved in H₂O (50 mL). To the solution was added MeOH (50 mL). The mixture was then cooled to −10° C. A solution of compound 4a (19.0 g, 61.2 mL) in THF (300 mL) was added. The reaction mixture was allowed to warm to 15° C. over a period of 4 h. A saturated NH₄Cl solution (200 mL) and EtOAc (200 mL) were added. The organic phase was separated and the aqueous layer was extracted with EtOAc (200 mL). The combined organic extracts were washed with brine (2×200 mL), dried over Na₂SO₄, filtered and concentrated in vacuo to give the crude compound 4 which was crystallized from a mixture of EtOAc and hexane (1: 4) to give the pure compound 5a as a off white needles (12.1 g, 85% yield). ¹H NMR (500 MHz, CDCl₃) δ 2.96 (bs, 1H), 4.29 (s, 2H), 4.56 (s, 2H), 7.30 (t, J=5.0 Hz, 1H), 7.44 (t, J=5.0 Hz, 2H), 8.33 (bs, 1H); Anal. Calcd for C₉H₉Cl₂NO₂: C, 46.18; H, 3.87; N, 5.98. Found: C, 46.21; H, 3.72; N, 5.80.

### D. Preparation of N-(2-chloro-6-(hydroxymethyl)phenyl)-2-(tritylamino)thiazole-5-carboxamide (7a)

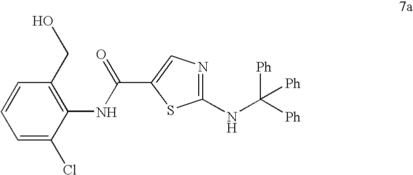

A mixture of compound 5a (5.85 g, 25 mmol) and compound 6a (9.34 g, 25 mmol) in MeOH (50 mL) was heated to reflux for 22 h. The reaction mixture was then cooled to −10° C. The solid was filtered and washed with cold MeOH (2×10 mL). The filter cake was vacuum air dried overnight to give compound 7a (10.0 g, 76% yield). ¹H NMR (500 MHz, DMSO) δ 4.40 (d, J=5.0 Hz, 2H), 5.23 (t, J=5.0 Hz, 1H), 7.22-7.48 (m, 18H), 7.69 (s, 1H), 9.18 (s, 1H), 9.56 (s, 1H).

### E. Preparation of 3-chloro-2-(2-(tritylamino)thiazole-5-carboxamido)benzyl pivalate (9a)

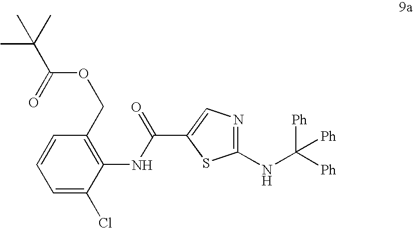

To a solution of compound 7a (7.36 g, 14 mmol) in THF (200 mL) at 5° C. were added N-methylmorpholine (3.1 mL, 28 mmol) followed by slow addition of trimethylacetyl chloride (8a, 1.8 mL, 21 mmol). The reaction mixture was allowed to warm to room temperature over a period of 3 h. A solution of saturated NH₄Cl in H₂O (500 mL) was added. The mixture was extracted with EtOAc (2×250 mL). The combined extracts were washed with 1N HCl (500 mL), saturated NaHCO₃ (500 mL), dried over MgSO₄. The solution was filtered and concentrated in vacuo to give compound 9a as a white solid (8.90 g, 100% yield). ¹H NMR (500 MHz, CDCl₃) δ 1.16 (s, 9H), 5.01 (s, 2H), 7.20-7.40 (m, 18H), 7.79 (s, 1H), 8.30 (bs, 1H).

### F. Preparation of 2-(2-aminothiazole-5-carboxamido)-3-chlorobenzyl pivalate (10a)

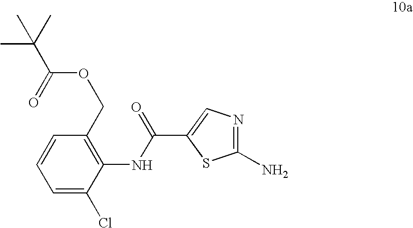

A mixture of compound 9a (8.90 g, 14 mmol) and formic acid (15 mL, 96% in H₂O) in EtOH (150 mL) was refluxed for 24 h. After cooling, the solution was concentrated in vacuo. The residue was subjected to a short column using EtOAc in hexane (30% to 70%) to give the crude compound 10a which was triturated with 10% EtOAc in hexane to give compound 10a (4.50 g, 87% yield). ¹H NMR (500 MHz, CDCl₃) δ 1.17 (s, 9H), 5.09 (s, 2H), 7.20-7.39 (m, 5H), 8.01 (s, 1H), 8.84 (s, 1H).

### G. Preparation of 3-chloro-2-(2-(6-chloro-2-methylpyrimidin-4-ylamino)thiazole-5-carboxamido)benzyl pivalate (12a)

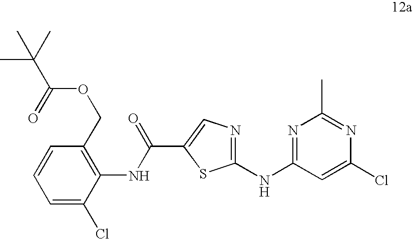

To a solution of compound 10a (2.21 g, 6 mmol) and 4,6-dichloro-2-methylpyrimidine (11a, 1.18 g, 7.2 mmol) in THF (30 mL) was added a solution of NaOBu-t in THF (30% w/w, 7.4 mL) at temperature between 10° C. to 18° C. The reaction mixture was stirred at 18° C. for 1.5 h. It was then cooled to 0° C. A solution of 1N HCl (14.0 mL) was added. The mixture then was partitioned between EtOAc and H₂O (1:1, 200 mL). The organic phase was separated, and the aqueous layer was extracted with EtOAc (100 mL). The combined extracts were concentrated in vacuo. The residue was subjected to a flash column using 50% EtOAc in hexane to give crude compound 12a which was triturated with 10% EtOAc in hexane to give compound 12a (1.95 g, 66% yield). ¹H NMR (500 MHz, CDCl₃) δ 1.19 (s, 9H), 2.73 (s, 3H), 5.12 (s, 2H), 6.93 (s, 1H), 7.21-7.49 (m, 3 H), 8.21 (s, 1H), 8.98 (1H).

### H. Preparation of 2-(6-chloro-2-methylpyrimidin-4-ylamino)-N-(2-chloro-6-(hydroxymethyl)phenyl)thiazole-5-carboxamide (13a)

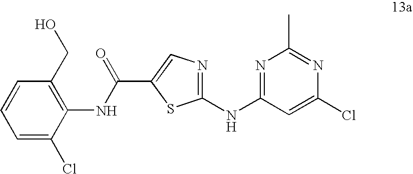

To a solution of lithium hydroxide mono hydrate in H₂O (4 mL) were added MeOH (8 mL), compound 12a (1.93 g, 3.9 mmol) and THF (32 mL) respectively. The reaction mixture was stirred at room temperature for 6 h. To the reaction mixture was added a solution of 1N HCl (38 mL) to adjust the PH=7. H₂O (20 mL) was added. The mixture was stirred at room temperature for 30 mins. The precipitates were filtered and the filter cake was washed with H₂O (3×10 mL) and then vacuum air dried overnight. Compound 13a was collected as white solid (1.57 g, 98% yield). ¹H NMR (500 MHz, DMSO-d₆) δ 2.60 (s, 3H), 4.50 (d, J=5.0 Hz, 2H), 5.31 (t, J=5.0 Hz, 1H), 6.96 (s, 1H), 7.39 (t, J=5.0 Hz, 1H), 7.47 (d, J=5.0 Hz, 1H), 7.53 (d, J=5.0 Hz, 1H), 8.31 (s, 1H), 9.95 (s, 1H), 12.24 (s, 1H).

### Example 1

### Preparation of N-(2-chloro-6-(hydroxymethyl)phenyl)-2-(6-(4-(2-hydroxyethyl)piperazin-1-yl)-2-methylpyrimidin-4-ylamino)thiazole-5-carboxamide (II):

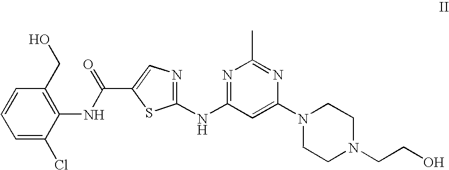

To a 20 mL microwave reaction tube was charged compound 13a (688 mg, 1.68 mmol), 2-hydroxyethylpiperizine (14, 437 mg, 3.36 mmol), ethyldiisopropylamine (434 mg, 3.36 mmol) and EtOH (10 mL). The mixture was heated at 110° C. for 1 h using a microwave reactor. The reaction mixture was then subjected to a short column using 0 to 10% 2M NH₃ in MeOH in CH₂Cl₂ as the eluent to give compound II as a white solid (504 mg, 60% yield). ¹H NMR (500 MHz, MeOD) δ 2.49 (s, 3H), 2.59-2.64 (m, 6H), 3.66-3.68 (t, J=5.2 Hz, 4H), 3.75 (t, J=6.0 Hz, 2H), 4.68 (s, 2H), 6.03 (s, 1H); 7.39 (t, J=7.7 Hz, 1H), 7.47 (d, J=7.7 Hz, 1H), 7.56 (d, J=7.7 Hz, 1H), 8.17 (s, 1H); Anal. Calcd for C₂₂H₂₆CN₇O₃S.0.57H₂O: C, 51.38; H, 5.32; N, 19.07. Found: C, 51.16; H, 5.22; N, 18.97.

### Example 2

### Preparation of N-(2-chloro-6-methylphenyl)-2-(6-(4-(2-hydroxyethyl)piperazin-1-yl)-2-methylpyrimidin-4-ylamino)thiazole-5-carboxamide (I)

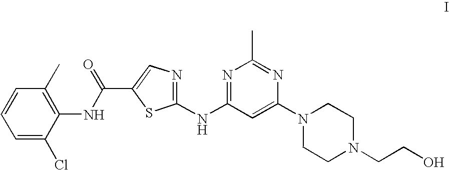

The title compound was prepared according to the following reaction scheme:

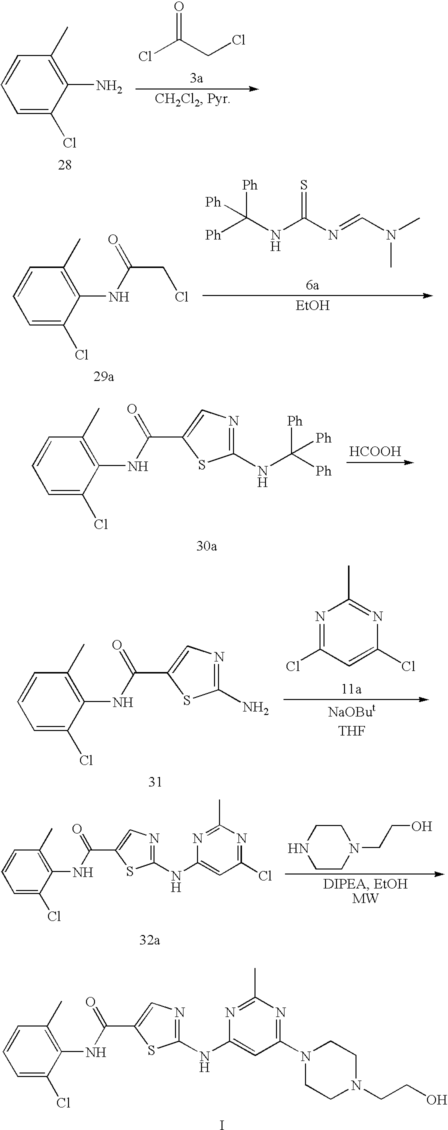

### A. Preparation of 2-chloro-N-(2-chloro-6-methylphenyl)acetamide (29a)

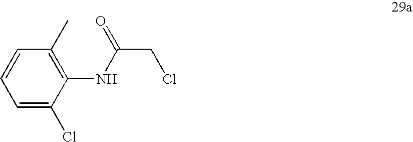

To a solution of 2-chloro-6-methyl-aniline (28) (25.0 g, 176.6 mmol) in CH₂Cl₂ (200 mL) was added pyridine (28.6 mL, 353 mmol) at −10° C. After 10 mins, chloroacetyl chloride (3a) (21.1 mL, 265 mmol) was added. The reaction mixture was allowed to warm to room temperature over a period of 2 h. A solution of 1 N HCl (500 mL) was added and the mixture was stirred for 10 mins. The organic phase was separated and the aqueous layer was extracted with CH₂Cl₂ (2×300 mL). The combined organic extracts were washed with 1 N HCl solution (400 mL), H₂O (400 mL) and brine (400 mL). It was dried over MgSO₄, filtered and concentrated in vacuo to give the product 29a as a white solid (37.8 g, 98% yield). ¹H NMR (500 MHz, CDCl₃) δ 2.28 (s, 3H), 4.25 (s, 2H), 7.14-7.18 (m, 2H), 7.26-7.31 (m, 1H), 8.02 (bs, 1H).

### B. Preparation of N-(2-chloro-6-methylphenyl)-2-(tritylamino)thiazole-5-carboxamide (30a)

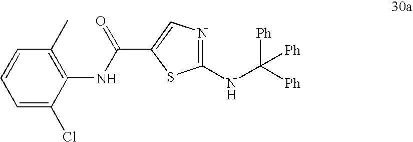

A mixture of compound 29a (1.09 g, 5 mmol) and compound (6a) (1.87 g, 5 mmol) in EtOH (25 mL) was heated to reflux for 6 h. LCMS confirmed the formation of compound 30a.

### C. Preparation of 2-amino-N-(2-chloro-6-methylphenyl)thiazole-5-carboxamide (31)

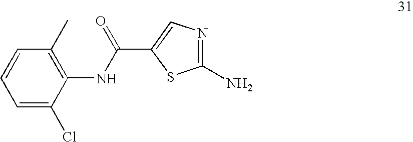

To the reaction solution obtained in the previous step was added 96% formic acid (5 mL). The reaction mixture was refluxed overnight. The reaction mixture was then partitioned between EtOAc and H₂O (1:1, 100 mL). The organic phase was separated and concentrated in vacuo. The residue was subjected to column chromatography using EtOAc in hexane (20% to 80%) to give the product 31 (0.94 g, 70% yield). ¹H NMR (300 MHz,DMSO-d₆) δ 2.21 (s, 3H), 7.20-7.22 (m, 2H), 7.37 (dd, J=7.2 and 2.4 Hz, 1H), 7.57 (s, 2H), 7.86 (s, 1H), 9.60 (s, 1H); LCMS m/z 268.00 (M+H).

### D. Preparation of 2-(6-chloro-2-methylpyrimidin-4-ylamino)-N-(2-chloro-6-methylphenyl)thiazole-5-carboxamide (32a)

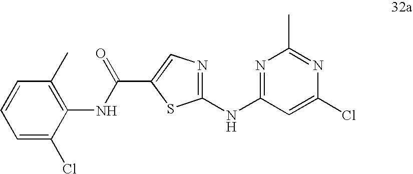

Compound 32a is prepared according to the procedure described in US/2006/0004067A1 (Bang-Chi Chen, et al, published Jan. 05, 2006).

### E. N-(2-chloro-6-methylphenyl)-2-(6-(4-(2-hydroxyethyl)piperazin-1-yl)-2-methylpyrimidin-4-ylamino)thiazole-5-carboxamide (I)

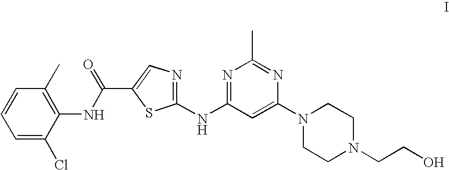

Compound I is prepared according to the procedure described in US/2006/0004067A1 (Bang-Chi Chen, et al, published Jan. 05, 2006).

### Example 3

### Preparation 2-(4-(6-(5-(2-chloro-6-methylphenylcarbamoyl)thiazol-2-ylamino)-2-methylpyrimidin-4-yl)piperazin-1-yl)acetic acid (IV)

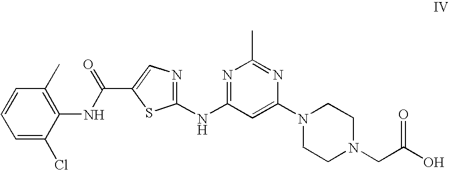

The title compound was prepared according to the following reaction scheme:

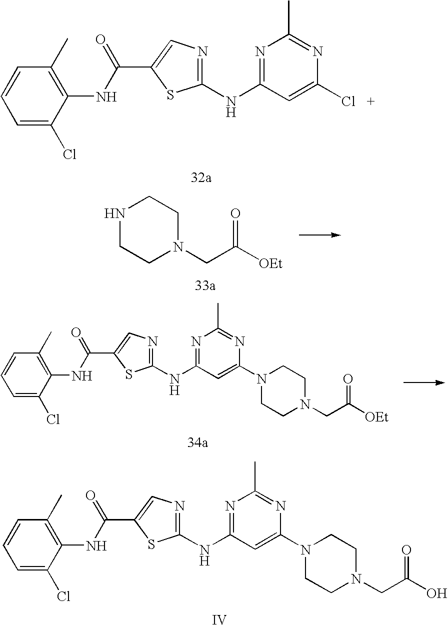

### A. Preparation of ethyl 2-(4-(6-(5-(2-chloro-6-methylphenylcarbamoyl)thiazol-2-ylamino)-2-methylpyrimidin-4-yl)piperazin-1-yl)acetate (34a)

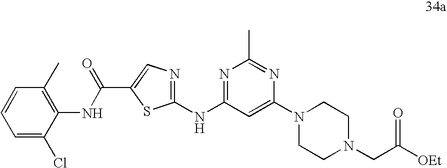

A mixture of 2-(6-chloro-2-methylpyrimidin-4-ylamino)-N-(2-chloro-6-methylphenyl)thiazole-5-carboxamide (32a) (0.20 g, 0.51 mmol) and 1-(ethoxycarbonylmethyl)piperazine (33) (0.17 g, 1.01 mmol) in 4 mL of EtOH was heated with a CEM Discover® microwave at 250 W, Pmax 150° C. for 45 min. The resulting mixture was treated with EtOH (6 mL) and then stirred at rt for 1 h. The solid was collected on a filter to give 0.24 g of ethyl 2-(4-(6-(5-((2-chloro-6-methylphenyl)carbamoyl)thiazol-2-ylamino)-2-methylpyrimidin-4-yl)piperazin-1-yl)acetate (34a) (89% yield). HPLC>99% pure (Rt 2.517 min); LC/MS (ES⁻) 530/532 (M+H, 100); ¹H NMR (DMSO-d6) δ 11.48 (s, 1H), 9.86 (s, 1H), 8.22 (s, 1H), 7.40 (m, 1H), 7.28 (m, 2H), 6.06 (s, 1H), 4.10 (q, J=7.1 Hz, 2H), 3.53 (s, 4H), 3.29 (s, 2H), 2.59 (t, J=4.6 Hz, 4H), 2.41 (s, 3H), 2.24 (s, 3H), 1.20 (t, J=3H); Anal. Calcd for C₂₄H₂₈ClN₇O₃S: C, 54.38; H, 5.32; Cl, 6.69; N, 18.50; S, 6.05. Found:: C, 54.24; H, 5.16; Cl, 6.80; N, 18.59; S, 6.09.

HPLC conditions column: YMC ODS-A S5 4.6×50 mm; UV: 220 nm; gradient time: 4 min; flow rate: 4 mL/min, 0-100% B; solvent A: 10% MeOH/90% H₂O with 0.2% H₃PO₄, solvent B: 90% MeOH/10% H₂O with 0.2% H₃PO₄.

### B. Preparation of 2-(4-(6-(5-(2-chloro-6-methylphenylcarbamoyl)thiazol-2-ylamino)-2-methylpyrimidin-4-yl)piperazin-1-yl)acetic acid (IV)

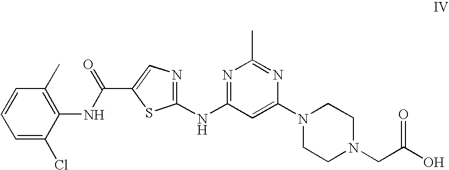

A mixture of ethyl 2-(4-(6-(5-((2-chloro-6-methylphenyl)carbamoyl)thiazol-2-ylamino)-2-methylpyrimidin-4-yl)piperazin-1-yl)acetate (34a) (0.21 g, 0.40 mmol) in 10 mL of MeOH was added 1.0 N NaOH (2.4 mL, 2.4 mmol) at rt. The mixture was stirred at rt for 1 h to become a clear solution. The HPLC indicated the reaction was complete with a single peak with Rt 2.417 min. The solvents in the solution were reduced to a small volume. The remaining liquid was treated with H₂O (10 mL). The aqueous solution was washed with CH₂Cl₂ (2×10 mL) and then adjusted to pH 6-7 with 1 N HCl (2.4 mL) to result in white precipitation. The solid was collected on a filter, washed with water to give 0.18 g of 2-(4-(6-(5-((2-chloro-6-methylphenyl)carbamoyl)thiazol-2-ylamino)-2-methylpyrimidin-4-yl)piperazin-1-yl)acetic acid (IV) (90% yield). HPLC>99% pure. LC/MS (ES⁺) 502/504 (M+H, 100), HRMS calcd for C₂₂H₂₄ClN₇O₃S 502.1428, found 502.1413. 1H NMR (DMSO-d6) δ 11.49 (br s, 1H), 9.89 (s, 1H), 8.23 (s, 1H), 7.41(dd, J=7.5, 1.6 Hz, 1H), 7.28 (m, 2H), 6.07 (s, 1H), 3.55 (s, 4H), 3.19 (s, 2H), 2.61 (t, J=4.7 Hz, 4H), 2.42 (s, 3H), 2.24 (s, 3H). Anal. Calcd for C₂₂H₂₄ClN₇O₃S 0.85 H₂O: C, 51.08; H, 5.01; Cl, 6.85; N, 18.95; S, 6.20; H₂O, 2.96%. Found: C, 51.23; H, 4.97; Cl, 7.08; N, 19.09; S, 5.92; H₂O, 2.94%.

What is claimed is:

1. A process for preparing a compound of formula (Ia) 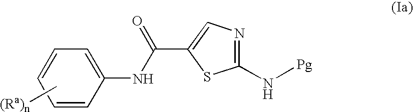
wherein R^{a} is selected from H, alkyl, halo, alkoxyl, C(O)OH, C(O)O-alkyl, alkyl-OC(O)—R¹, hydroxyalkyl and/or suitable protecting groups thereof,
n is selected from 0, 1, 2 and 3;
R¹ is selected from hydrogen, alkyl and aryl;
including the steps of reacting a compound of formula 6 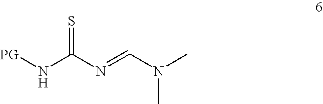
where PG is a nitrogen protecting group, with a compound of formula (A) 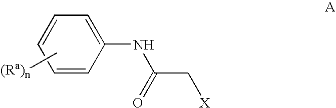
wherein X is selected from Cl, Br, I, mesylate, and tosylate;
to provide the compound of formula (Ia).

2. A process for preparing a compound of formula (IIa), 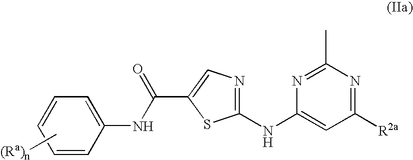
wherein
R^{a} is selected from H, alkyl, halo, alkoxyl, C(O)OH, C(O)O-alkyl, alkyl-OC(O)—R¹, hydroxyalkyl, and/or suitable protecting groups thereof,
n is selected from 0, 1, 2 and 3;
R¹ is selected from hydrogen, alkyl and aryl;
R^{2a} is selected from halo, 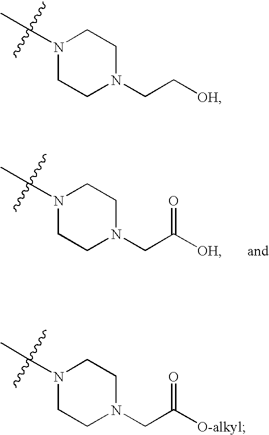
including the steps of reacting a compound of formula 6 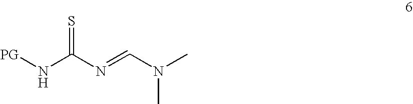
where PG is a nitrogen protecting group, with a compound of formula (A) 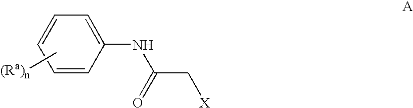
wherein X is selected from halo, mesylate, and tosylate;
to provide the compound of formula (Ia) 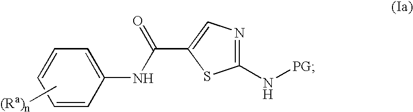
reacting the compound of formula Ia
with a deprotecting group to remove the Pg, followed by reaction with a compound of formula 2a 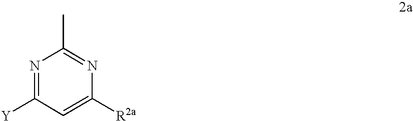
wherein Y is selected from Cl, Br, and I,
to give the compound of formula IIa.

3. The process of claim 2, wherein the compound of formula IIa has the formula (IIa′)

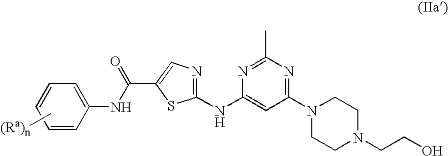

4. The process of claim 2, wherein
the compound of formula (A) is selected from

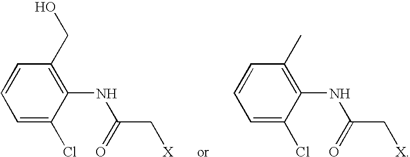

5. The process of claim 4, wherein
the compound of formula IIa has the formula II; 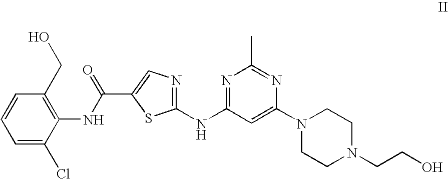
the process including the steps of
a) reacting the compound of formula 4 with a base to give a compound of formula 5 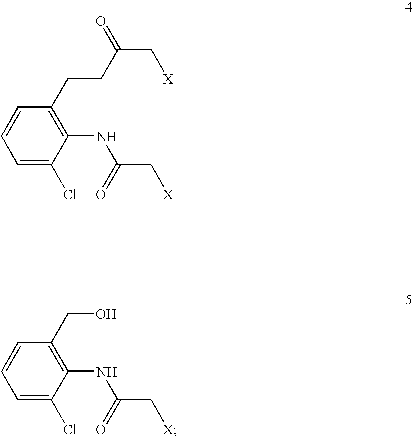
b) reacting the compound of formula 5 with a compound of formula 6 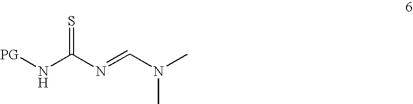
to give a compound of formula 7 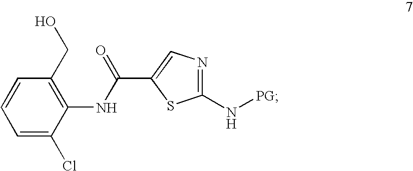
c) reacting the compound of formula 7 with a compound of formula 8 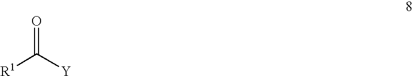
where Y is selected from Cl, Br, and I, and R¹ is selected from hydrogen, alkyl and aryl, to give a compound of formula 9 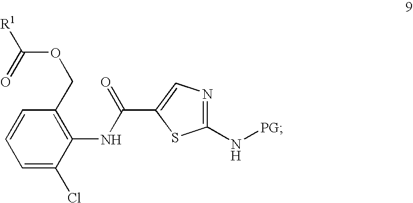
d) treating the compound of formula 9 with a deprotecting reagent to give a compound of formula 10 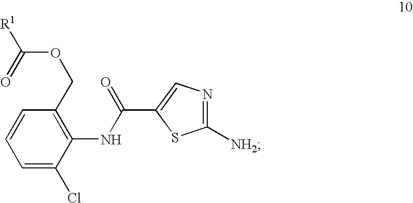
e) reacting the compound of formula 10 with a compound of formula 11 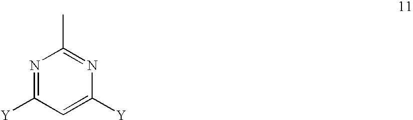
wherein Y is selected from Cl, Br, and I,
in the presence of a base to give compound of formula 12 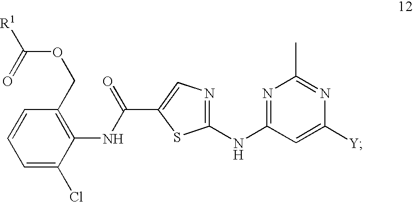
f) reacting the compound of formula 12 with a base to give compound of formula 13 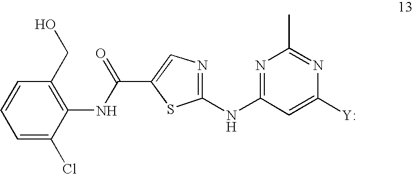
g) and reacting the compound of formula 13 with a compound of formula 14 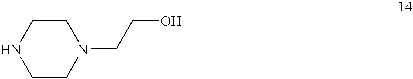
in the presence of a base and to give compound of formula II

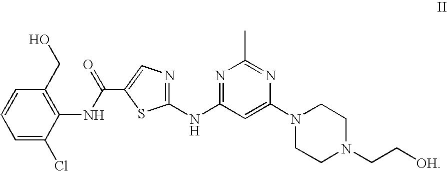

6. The process of claim 4, wherein
the compound formula IIa has the formula I; 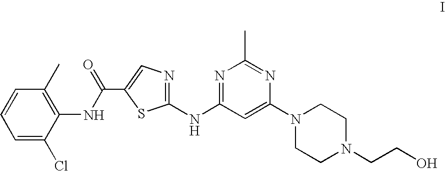
including the steps of
a) reacting the compound of formula 29 with a compound of formula 6 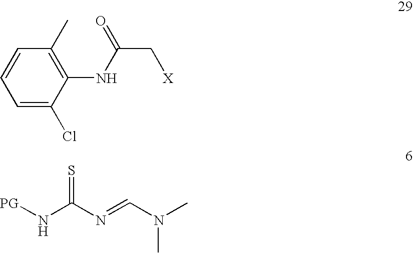
to give a compound of formula 30; 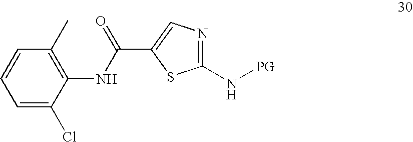
c) treating the compound of formula 30 with a deprotecting reagent to give a compound of formula 31 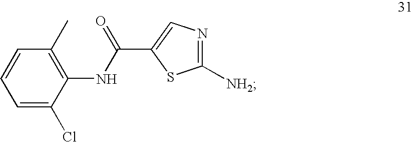
d) reacting the compound of formula 31 with a compound of formula 11 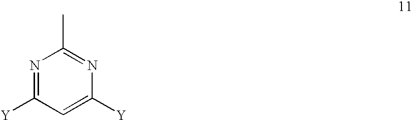
to give a compound of formula 32 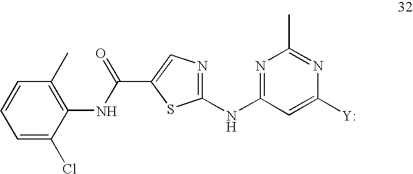
e) and reacting the compound of formula 32 with a compound of formula 14 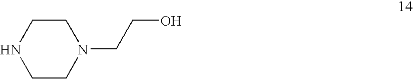
in the presence of a base conditions to give compound of formula I

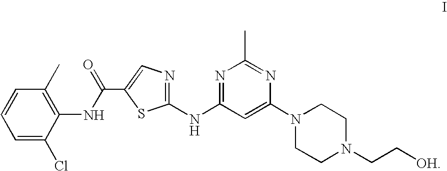

7. The process of claim 4, wherein
the compound of formula IIa has the formula II; 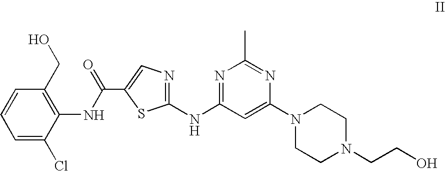
the process includes the steps of
a) reacting a compound with a formula 1 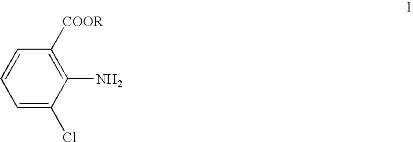
where R is selected from hydrogen, alkyl and aryl, with a reducing reagent to give a compound of formula 2 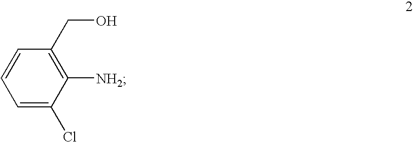
b) reacting the compound of formula 2 with a compound of formula 3 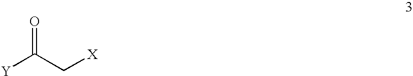
wherein Y is selected from Cl, Br, and I, in the presence of a base to give a compound of formula 4 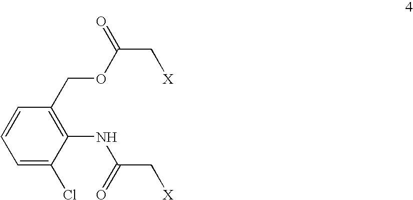
where X is as defined above;
c) reacting the compound of formula 4 with a base to give a compound of formula 5; 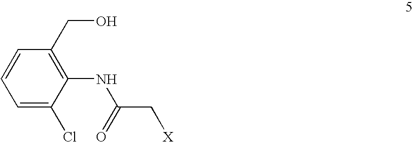
d) reacting the compound of formula 5 with a compound of formula 6 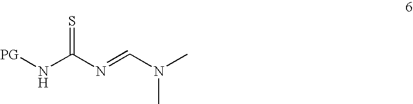
to give a compound of formula 7; 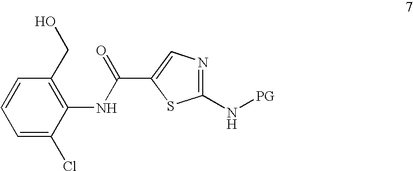
e) reacting the compound of formula 7 with a compound of formula 8 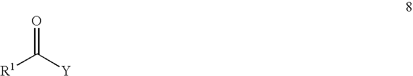
and R¹ is selected from hydrogen, alkyl and aryl, to give a compound of formula 9; 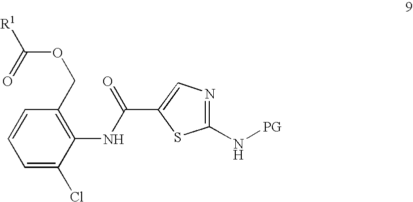
g) treating the compound of formula 9 with a deprotecting reagent to give a compound of formula 10; 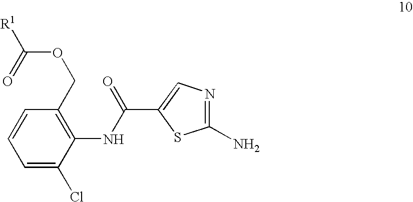
h) reacting the compound of formula 10 with a compound of formula 11 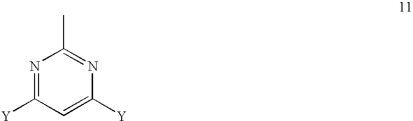
in the presence of a base to give compound of formula 12; 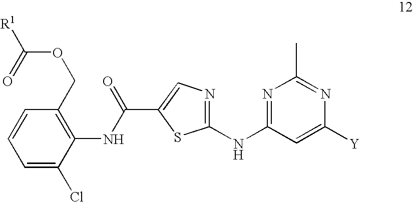
i) reacting the compound of formula 12 with a base to give compound of formula 13; 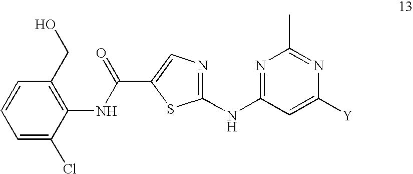
j) and reacting the compound of formula 13 with a compound of formula 14 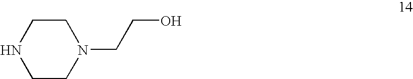
in the presence of a base and to give compound of formula II

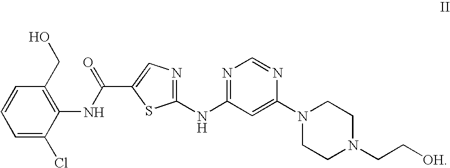

8. The process of claim 4, wherein
the compound formula IIa has the formula I; 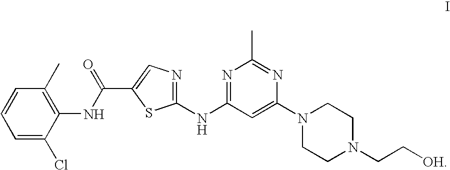
including the steps of
a) reacting the compound of formula 28 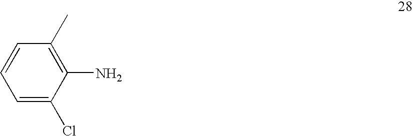
with a compound of formula 3 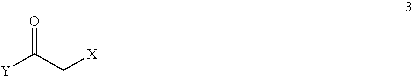
wherein Y is selected from Cl, Br, and I, in the presence of a base to give a compound of formula 29 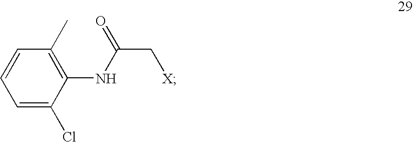
b) reacting the compound of formula 29 with a compound of formula 6 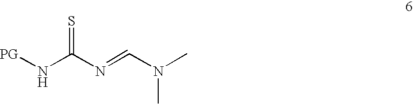
to give a compound of formula 30; 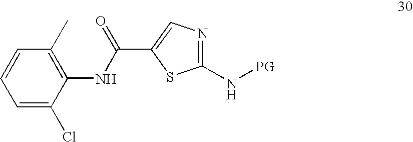
c) treating the compound of formula 30 with a deprotecting reagent to give a compound of formula 31 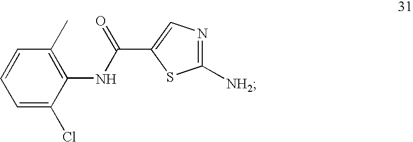
d) reacting the compound of formula 31 with a compound of formula 11 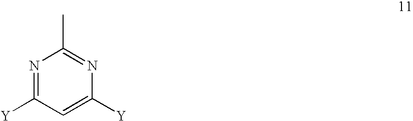
to give a compound of formula 32 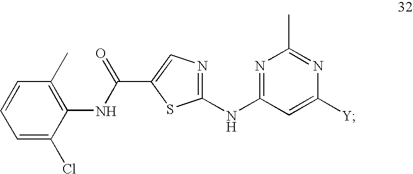
e) and reacting the compound of formula 32 with a compound of formula 14 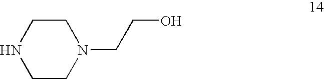
in the presence of a base to give compound of formula I

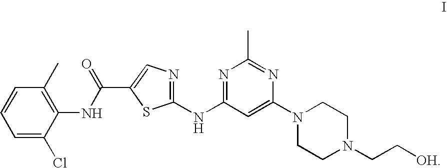

9. The process of claim 2, wherein the compound of formula IIa has the formula IV 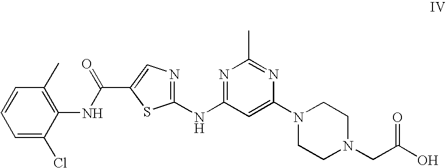
which includes the steps of
a) reacting the compound of formula 32 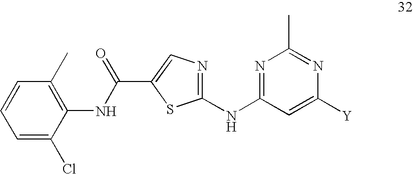
where Y is selected from Cl, Br, and I, with a compound of formula 33 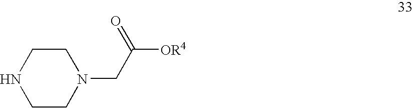
where R⁴ is selected from hydrogen, alkyl and aryl, to give a compound of formula 34 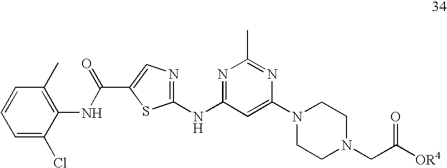
where R⁴ is as defined above;
b) treating the compound of formula 34 with a base to give compound of formula IV

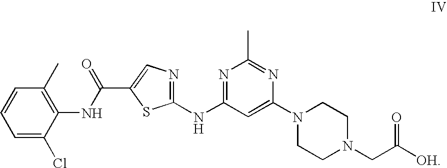

10. The process of claim 1, wherein
Pg is selected from benzyl, p-methoxybenzyl, diphenylmethyl and trityl; and
the step of reacting the compound of formula 6 with the compound of formula (A) is done in a solvent selected from MeOH, EtOH, and Pr—OH.

11. The process of claim 1, wherein R^{a} is selected from hydrogen alkyl, halo, hydroxyalkyl, C(O)OH, C(O)O-alkyl, alkyl-OC(O)—R¹.

12. The process of claim 2, wherein
Pg is selected from benzyl, p-methoxybenzyl, diphenylmethyl and trityl; and
the step of reacting the compound of formula 6 with the compound of formula (A) is done in a solvent selected from MeOH, EtOH, and Pr—OH.
